SEQUENCE LISTING (60 sequences)

SEQ ID NO: 1 - protein, 12 aa, Homo sapiens
EQFLDGDGWTSR

SEQ ID NO: 2 - protein, 13 aa, Homo sapiens
HEQNIDCGGGYVK

SEQ ID NO: 3 - protein, 8 aa, Homo sapiens
QIDNPDYK

SEQ ID NO: 4 - protein, 10 aa, Homo sapiens
SPAHYAGYWK

SEQ ID NO: 5 - protein, 8 aa, Homo sapiens
AVQELWLR

SEQ ID NO: 6 - protein, 10 aa, Homo sapiens
DTLENTIGHR

SEQ ID NO: 7 - protein, 10 aa, Homo sapiens
  MOD_RES (4)..(4): M[oxi]
DAGMQLQGYR

SEQ ID NO: 8 - protein, 10 aa, Homo sapiens
DAGMQLQGYR

SEQ ID NO: 9 - protein, 8 aa, Homo sapiens
TQLVSNLK

SEQ ID NO: 10 - protein, 10 aa, Homo sapiens
GEFVTTVQQR

SEQ ID NO: 11 - protein, 12 aa, Homo sapiens
FVEGLPINDFSR

SEQ ID NO: 12 - protein, 12 aa, Homo sapiens
  MOD_RES (8)..(8): M[oxi]
IHQIEYAMEAVK

SEQ ID NO: 13 - protein, 12 aa, Homo sapiens
IHQIEYAMEAVK

SEQ ID NO: 14 - protein, 11 aa, Homo sapiens
FVFDRPLPVSR

SEQ ID NO: 15 - protein, 9 aa, Homo sapiens
DEAAYGELR

SEQ ID NO: 16 - protein, 16 aa, Homo sapiens
AFYAELYHIISSNLEK

SEQ ID NO: 17 - protein, 15 aa, Homo sapiens
NYTDEAIETDDLTIK

SEQ ID NO: 18 - protein, 9 aa, Homo sapiens
ILNPEEIEK

SEQ ID NO: 19 - protein, 7 aa, Homo sapiens
LAVYIDK

SEQ ID NO: 20 - protein, 11 aa, Homo sapiens
DAALATALGDK

SEQ ID NO: 21 - protein, 9 aa, Homo sapiens
IGDTSVSYK

SEQ ID NO: 22 - protein, 20 aa, Homo sapiens
LPFTALGSGQDAALAVLEDR

SEQ ID NO: 23 - protein, 10 aa, Homo sapiens
TLSSPTEPVK

SEQ ID NO: 24 - protein, 13 aa, Homo sapiens
VIGGDDLSTLTGK

SEQ ID NO: 25 - protein, 13 aa, Homo sapiens
NVLIVEDIIDTGK

SEQ ID NO: 26 - protein, 15 aa, Homo sapiens
THYSNIEANESEEVR

SEQ ID NO: 27 - protein, 7 aa, Homo sapiens
LGFEEFK

SEQ ID NO: 28 - protein, 7 aa, Homo sapiens
YLWNNIK

SEQ ID NO: 29 - protein, 12 aa, Homo sapiens
GFQALGDAADIR

SEQ ID NO: 30 - protein, 9 aa, Homo sapiens
SEEFLIAGK

SEQ ID NO: 31 - protein, 7 aa, Homo sapiens
HLACLPR

SEQ ID NO: 32 - protein, 7 aa, Homo sapiens
YQEALAK

SEQ ID NO: 33 - protein, 14 aa, Homo sapiens
GDVTAQIALQPALK

SEQ ID NO: 34 - protein, 8 aa, Homo sapiens
LEEQRPER

SEQ ID NO: 35 - protein, 7 aa, Homo sapiens
HILANFK

SEQ ID NO: 36 - protein, 7 aa, Homo sapiens
DGLEMEK

SEQ ID NO: 37 - protein, 7 aa, Homo sapiens
  MOD_RES (5)..(5): M[oxi]
DGLEMEK

SEQ ID NO: 38 - protein, 8 aa, Homo sapiens
SIISMFDR

SEQ ID NO: 39 - protein, 8 aa, Homo sapiens
  MOD_RES (5)..(5): M[oxi]
SIISMFDR

SEQ ID NO: 40 - protein, 10 aa, Homo sapiens
YITDWQNVFR

SEQ ID NO: 41 - protein, 11 aa, Homo sapiens
LSDQFHDILIR

SEQ ID NO: 42 - protein, 10 aa, Homo sapiens
VSAGEAVVNR

SEQ ID NO: 43 - protein, 21 aa, Homo sapiens
DGSSGGVIYLVTITAAGVDHR

SEQ ID NO: 44 - protein, 11 aa, Homo sapiens
LAAIAESGVER

SEQ ID NO: 45 - protein, 10 aa, Homo sapiens
QVLLGDQIPK

SEQ ID NO: 46 - protein, 10 aa, Homo sapiens
VAASNIVQMK

SEQ ID NO: 47 - protein, 10 aa, Homo sapiens
  MOD_RES (9)..(9): M[oxi]
VAASNIVQMK

SEQ ID NO: 48 - protein, 11 aa, Homo sapiens
FILNLPTFSVR

SEQ ID NO: 49 - protein, 14 aa, Homo sapiens
GDGPVQGIINFEQK

SEQ ID NO: 50 - protein, 12 aa, Homo sapiens
HVGDLGNVTADK

SEQ ID NO: 51 - protein, 10 aa, Homo sapiens
LACGVIGIAQ

SEQ ID NO: 52 - protein, 7 aa, Homo sapiens
EHYVDLK

SEQ ID NO: 53 - protein, 10 aa, Homo sapiens
DRPFFAGLVK

SEQ ID NO: 54 - protein, 14 aa, Homo sapiens
NIIHGSDSVESAEK

SEQ ID NO: 55 - protein, 10 aa, Homo sapiens
DRPFFPGLVK

SEQ ID NO: 56 - protein, 10 aa, Homo sapiens
NIIHGSDSVK

SEQ ID NO: 57 - protein, 15 aa, Homo sapiens
EISLWFKPEELVDYK

SEQ ID NO: 58 - protein, 14 aa, Homo sapiens
TQSSLVPALTDFVR

SEQ ID NO: 59 - protein, 27 aa, Homo sapiens
SVHPYEVAEVIALPVEQGNFPYLQWVR

SEQ ID NO: 60 - protein, 14 aa, Homo sapiens
QVTESVSDSITVLP

(19) United States
(12) Patent Application Publication
(10) Pub. No.: US 2014/0256586 A1
(43) Pub. Date: Sep. 11, 2014

(54) METHODS AND COMPOSITIONS FOR DIAGNOSIS OF COLORECTAL CANCER

(71) Applicant: The Wistar Institute of Anatomy and Biology, Philadelphia, PA (US)

(72) Inventors: David W. Speicher, Berwyn, PA (US); Wona Joo, Wayne, PA (US)

(73) Assignee: The Wistar Institute of Anatomy and Biology, Philadelphia, PA (US)

(21) Appl. No.: 14/201,193

(22) Filed: Mar. 7, 2014

Related U.S. Application Data
(60) Provisional application No. 61/775,501, filed on Mar. 9, 2013.

(51) Int. Cl. G01N 33/574

(52) U.S. Cl. CPC G01N 33/57419
USPC 506/9; 530/391.3; 506/18; 536/24.31; 435/287.2; 435/7.92; 435/7.94; 435/6.12; 435/6.11

(57) ABSTRACT

Methods and compositions are provided for diagnosing colorectal cancer in a mammalian subject, preferably in a serum or plasma sample of a human subject. The methods and compositions enable the detection or measurement in the sample or from a protein level profile generated from the sample, the protein level of one or more specified biomarkers. Comparing the protein level(s) of the biomarker(s) in the subject's sample or from protein abundance profile of multiple biomarkers, with the level of the same biomarker(s) or profile in a reference standard, permits the determination of a diagnosis of colorectal cancer, or the identification of a risk of developing colorectal cancer, or enables the monitoring of the status of progression or remission of colorectal cancer in the subject followed during a therapeutic protocol.

### STATEMENT REGARDING FEDERALLY SPONSORED RESEARCH OR DEVELOPMENT

This invention was made with government support under Grant Nos. CA120393 awarded by the National Institutes of Health. The government has certain rights in this invention.

### BACKGROUND OF THE INVENTION

Despite advances in understanding the genetic pathways involved in colorectal cancer (CRC) malignancies, improved treatments and progress in screening, CRC still remains the third leading cause of cancer-related deaths in the United States. Approximately 143,000 new cases occur annually and the lifetime risk of developing colorectal cancer is about 1 in 20 (5.1%). Most cases of CRC are sporadic resulting from the accumulation of somatic genetic abnormality and are associated with a variety of environmental risk factors. The high mortality and morbidity associated with CRC is mostly attributed to the inability to detect the disease at early stage before it is widely disseminated. Patients are usually diagnosed in advanced stage and only 5% of patients survive longer than five years. However, when CRC is detected and treated at an early stage, the five-year survival rate is greater than 74%.

Biomarkers associated with CRC can promote early detection, establish prognosis and help predict the response of the patient to specific therapies. The discovery of biomarkers also aids in the understanding of the biological mechanisms underlying disease development and progression. Currently, the most widely used tests for detection of CRC are fecal occult blood test (FOBT) and colorectal endoscopy. However, these tests have limitations. For example, with colorectal endoscopy, there is a lack of adequate screening of high risk groups due to the highly invasive nature of the test and the high expense associated therewith. DNA-based tests of blood or stool can have lower cost and are minimally invasive. However, the currently known CRC serological tumor markers, CEA and CA19-9, are used only to screen high risk patients and are not effective at detecting early stages of cancer. Hence, novel serological biomarkers are required to both improve early detection of CRC and assist in clinical management of the disease.

Recent advances in proteomics methods and availability of state-of-the-art mass spectrometers have improved the feasibility of identifying novel biomarkers. Typically investigators have studied established CRC cell lines (e.g. HCT116 and Smad-4) or lab developed low passage cell lines using 2D gel methods prior to MS analysis. In some cases, secretome or CRC mouse models have also been used. These studies were limited in the detection of low-abundance proteins, potential candidates for high-quality CRC biomarkers, due to the inherent limitations of a 2-D gel.

The use of a xenograft mouse model combined with 4-D protein profiling has been shown to facilitate detection of ovarian cancer serological biomarkers (Tang, H. Y. et al, A xenograft mouse model coupled with in-depth plasma proteome analysis facilitates identification of novel serum biomarkers for human ovarian cancer. J Proteome Res 2012, 11, (2), 678-91). The xenograft mouse model can minimize genetic, physiological and environmental variations inherent in human samples. In addition, higher concentrations of human protein are found in mouse blood due to the small blood volume, which allows for the detection of low-abundance protein. In addition, secretome analysis has also been shown to be a valuable method for detection of cancer biomarkers, due to the lower complexity of the proteome as compared to total cell lysate or plasma.

### SUMMARY OF THE INVENTION

In one aspect, a diagnostic reagent is provided which includes at least one ligand capable of specifically complexing with, binding to, or quantitatively detecting or identifying a single target biomarker selected from lamin B1 (LMNB1); enoyl-CoA delta isomerase 1 (ECI1, DCI); proteasome (prosome, macropain) subunit, beta type 6 (PSMB6); proteasome (prosome, macropain) subunit, beta type 9 (large multifunctional peptidase 2) (PSMB9); proteasome (prosome, macropain) subunit, alpha type, 1 (PSMA1); and an isoform, pro-form, modified molecular form, or peptide fragment of any of the above biomarkers, proteins in the same biomarker family or expressed from a related gene, having at least 20% sequence homology or sequence identity with any of the above biomarkers. At least one ligand is associated with a detectable label or with a substrate.

In another aspect, a diagnostic reagent is provided, which includes, in addition to at least one ligand described above, at least one additional ligand capable of specifically complexing with, binding to, or quantitatively detecting or identifying a single target biomarker selected from NME/NM23 nucleoside diphosphate kinase 2 (NME2); calreticulin (CALR); tumor protein, translationally-controlled 1 (TPT1); NME/NM23 nucleoside diphosphate kinase 1 (NME1); proteasome (prosome, macropain) subunit, alpha type, 7 (PSMA7); and an isoform, pro-form, modified molecular form, or peptide fragment of any of the above biomarkers, proteins in the same biomarker family or expressed from a related gene, having at least 20% sequence homology or sequence identity with any of the above biomarkers. At least one ligand is associated with a detectable label or with a substrate. In one embodiment, the reagent includes multiple ligands, each ligand directed to a different biomarker.

In another aspect, a reagent is provided which includes, in addition to one or more ligands described above, at least one ligand that specifically complexes with, binds to, quantitatively detects or identifies the biomarker, Carcinoembryonic Antigen (CEA), or an isoform, pro-form, modified molecular form, or peptide fragment therefrom.

In another aspect, a reagent is provided which includes, in addition to one or more ligands described above, at least one ligand that specifically complexes with, binds to, quantitatively detects or identifies the biomarker, Carbohydrate Antigen 19-9 (CA19-9), or an isoform, pro-form, modified molecular form, or peptide fragment thereof.

In another aspect, a reagent is provided which includes, in addition to one or more ligands described above, at least one ligand that specifically complexes with, binds to, quantitatively detects or identifies the biomarker, tissue inhibitor of metalloproteinases-1 (TIMP-1), or an isoform, pro-form, modified molecular form, or peptide fragment thereof.

In another aspect, a diagnostic kit, panel or microarray is provided. The kit, panel or microarray includes at least two diagnostic reagents described above, each reagent identifying a different biomarker.

In another aspect, a method for diagnosing or detecting or monitoring the progress of colorectal cancer in a subject is provided. The method includes contacting a sample obtained from a test subject with any of the diagnostic reagents described above; detecting or measuring in the sample or from a protein level profile generated from the sample, the protein levels of one or more of the biomarkers described above, or ratios thereof. The detection or measurement allows comparison of the protein levels of the biomarker in the subject's sample or from a protein level profile or ratio of multiple biomarkers, with the level of the same biomarker or biomarkers in a reference standard. In one embodiment, a significant change in protein level of the subject's sample biomarker or biomarkers from that in the reference standard indicates a diagnosis, risk, or the status of progression or remission of colorectal cancer in the subject.

In another aspect, a diagnostic reagent is provided for diagnosing, or detecting a risk of developing, colorectal cancer. The diagnostic reagent includes a ligand which is a nucleotide sequence capable of hybridizing to a nucleic acid sequence encoding any of the above described biomarkers. In one embodiment, the ligand is associated with a detectable label or with a substrate.

In yet another aspect, a method of diagnosing, or detecting a risk of developing, a colorectal cancer in a subject is provided. The method includes contacting a sample obtained from a test subject with any of the above-described diagnostic reagents and detecting or measuring in the sample or from an expression profile generated from the sample, the expression levels of one or more of the biomarkers described above, or ratios thereof. The detection or measurement allows for comparison of the expression levels of the biomarker in the subject's sample or from an expression level profile or ratio of multiple biomarkers, with the level of the same biomarker or biomarkers in a reference standard. In one embodiment, a significant change in expression level of the subject's sample biomarker or biomarkers from that in the reference standard indicates a diagnosis, risk, or the status of progression or remission of colorectal cancer in the subject.

In yet another aspect, a method for diagnosing or detecting or monitoring the progress of colorectal cancer in a subject is provided. The method includes (i) fragmenting proteins in a sample obtained from a test subject after contact with a chemical or enzymatic agent; (ii) injecting the digested sample of (i) into a mass spectrometer and identifying the protein levels of one or more of the above-described biomarkers, or ratios thereof, by mass spectrometry; and (iii) comparing the protein levels of the biomarker in the subject's sample with the level of the same biomarker or biomarkers in a reference standard. In one embodiment, a significant change in protein level of the subject's sample biomarker or biomarkers from that in the reference standard or from a predetermined cutoff indicates a diagnosis, risk, or the status of progression or remission of colorectal cancer in the subject. In one embodiment, the method further includes enriching the biomarker protein or one or more peptides produced by specific proteolysis in the sample by contacting the sample with an antibody prior to injecting into a mass spectrometer.

### BRIEF DESCRIPTION OF THE DRAWINGS

FIG. 1 is a table showing all quantitated proteins and peptides, linearity statistics, standard curve ranges, and ranges of concentrations observed in patients.

FIG. 2 is a schematic of the workflow used in this study, which compares the strategies for identification of serological cancer biomarker proteins from human colon cell line, WC013. Colon cancer cell line WC013 was used for the secretome and xenograft system. 2D_{—}44 method was performed by replicate of initial WC13 2D_{—}22 run and 2D_{—}88 run was done using two different passages secretome of WC013. After depletion of WC013 mouse plasma, sample was divided for 3D and 4D method. LC-MS/MS was conducted on individual pixels of each method (e.g. 4D_{—}175: total 175 pixels using 4D method).

FIGS. 3A-3C are gels showing protein separation of WC013 secretome and pre-fractionated WC013 plasma (4D) for GeLC-MS/MS. (a) For LC-MS/MS, each secretome from two different passages (P22 and P26) of WC013 was loaded onto gel and separated ˜2.2 cm in Secretome 2D method. (b) Right panel shows original undeleted plasma (P), major proteins bound affinity column (B) and resultant depletion plasma (D). Pre-fractionated of WC013 samples were separated in analytical gel after major plasma protein depletion. (c) For the maximum loading onto lane of gel, each fraction and membrane samples were concentrated and separated approximately 1 or 3 cm on gel. Number above lane of gel represents original plasma volume.

FIG. 4 is a bar graph showing a comparison of protein and peptide coverage for each method. Nonredundant protein counts from 2-D, 3-D, and 4-D data sets as shown. Peptide coverage of each method represents in bar. Two dimension eighty eight performed with biological duplicates of 2-D_{—}44 run as well as 3-D_{—}86 done with technical replicates of 3-D_{—}43 analysis. Total number of proteins identified was 3273, 408, 378, and 664 in 2-D_{—}88, 3-D_{—}86, 4-D_{—}85, and 4-D_{—}175, respectively.

FIG. 5 is a bar graph showing the reproducibility of MS analysis in secretome and xenograft methods. The identification from each replicate (2-D_{—}22, 2-D-44, and 3-D_{—}43) and different level of depth analysis (3-D_{—}86 vs 4-D_{—}85, 3-D_{—}86 vs 4-D_{—}175, and 4-D_{—}85 vs 4-D_{—}175) are compared to determine the overlap in identifications. All comparison performed with proteins identified by two and more nonredundant peptides at first level (P=2) and further comparison (Corrected P=2).

FIGS. 6A-6D are scatterplots for Group 1 candidate biomarkers. The group 1 proteins LMNB1 (a), ECI1 (b), NME2 (c) and CALR (d), show the largest difference between the 25 normal and 25 late cancer patients. Solid lines indicate the average of the samples shown within each group. “Low CEA” and “low CA19-9” data are levels of the indicated proteins observed in plasma from patients where the CEA or CA19-9 values were in the bottom half of advanced CRC patients. Grey data points indicate specimens where both CEA and CA19-9 were low. The statistical significance between normal subjects and these cancer subsets were comparable or better for the indicated biomarkers than the corresponding comparison for CEA and CA19-9 using these subsets. Asterisks (*) indicate significant difference (p<0.05) based on unpaired Mann-Whitney test with Welch's correction for comparison between normal and late cancer group and nonparametric test with Mann-Whitney test for comparison between normal and either low CEA or CA19-9.

FIGS. 7A-7D are scatterplots for Group 2 candidate biomarkers. The group 2 proteins, NME1 (a), TPT1 (b), CAPNS1 (c) and TIMP1 (d), show moderate, but non-significant differences between the normal and late cancer groups. TIMP1, the EDRN biomarker, was included for comparison. Solid lines indicate the average of the samples shown within each group. “Low CEA” and “low CA19-9” data are levels of the indicated proteins observed in plasma from patients where the CEA or CA19-9 values were in the bottom half of advanced CRC patients. Grey data points indicate specimens where both CEA and CA19-9 were low. Asterisks (*) indicate significant difference (p<0.05) based on unpaired Mann-Whitney test with Welch's correction for comparison between normal and late cancer group and nonparametric test with Mann-Whitney test for comparison between normal and either low CEA or CA19-9.

FIGS. 8A-8D are scatterplots of Group 3 candidate biomarkers. The group 3 proteins, PSMB6 (a), PSMB9 (b), PSMA1 (c) and PSMA (d), are proteosome subunits which are elevated in the low CEA late cancer specimens relative to normal donors and some of these differences are significant. Solid lines indicate the average of the samples shown within each group. “Low CEA” and “low CA19-9” data are levels of the indicated proteins observed in plasma from patients where the CEA or CA19-9 values were in the bottom half of advanced CRC patients. Grey data points indicate specimens where both CEA and CA19-9 were low. Asterisks (*) indicate significant difference (p<0.05) based on unpaired Mann-Whitney test with Welch's correction for comparison between normal and late cancer group and nonparametric test with Mann-Whitney test for comparison between normal and either low CEA or CA19-9.

FIGS. 9A-9I are scatterplots for Group 4 candidate biomarkers. These group 4 proteins, SDS1 (a), PDCD6 (b), PSMB10 (c), HPRT1 (d), MDH1 (e), PSME2 (f), SOD2 (g), PSMB2 (h) and CUT A (i), do not show any differences between groups and should be dropped. Solid lines indicate the average of the samples shown within each group. “Low CEA” and “low CA19-9” data are levels of the indicated proteins observed in plasma from patients where the CEA or CA19-9 values were in the bottom half of advanced CRC patients. Grey data points indicate specimens where both CEA and CA19-9 were low. Asterisks (*) indicate significant difference (p<0.05) based on unpaired Mann-Whitney test with Welch's correction for comparison between normal and late cancer group and nonparametric test with Mann-Whitney test for comparison between normal and either low CEA or CA19-9.

FIG. 10 is MRM ROC curves of selected candidate biomarkers of 25 normal and 25 late cancer samples. For reference, the AUC in this cohort was 0.78 for CEA and 0.74 for CA19-9.

FIG. 11 is a table showing the summary of biomarker analysis in 25 normal and 25 late CRC patients. The proteins shown above the dotted line have been shown to be valuable biomarkers of CRC. The proteins below the line do not warrant further testing.

### DETAILED DESCRIPTION OF THE INVENTION

The compositions and methods described herein provide means for diagnosing or detecting the existence or absence of, or monitoring the progress of, colorectal cancer in a subject using one or more of the biomarkers identified in Table 1 in optional combination with one or more known colorectal cancer-associated biomarkers.

Proteomics discovery of novel cancer plasma biomarkers is impeded by the vast complexity of plasma, variability of human specimens, and difficulty distinguishing cancer specific proteins from less specific plasma protein changes associated with the acute-phase inflammatory reaction. These hurdles were addressed by utilizing a xenograft mouse model coupled with an in-depth 4-D protein profiling method to identify human proteins in the mouse plasma.

Proteins confidently identified as human were definitely shed by the tumor into the blood and were therefore candidate serological cancer biomarkers. Parallel proteome analysis of cell culture conditioned medium (secretome) further enhanced sequence coverage of most proteins shed by the same human cell lines in the xenograft model. More than 800 human proteins were identified in a recently analyzed dataset from several low passage number colon carcinoma cell lines (WC013, WC007, WC020, and WC008), and 83 of these proteins were categorized as high priority candidate biomarkers. These candidates included novel candidate biomarkers as well as several proteins previously reported to be elevated in either CRC tumors or the blood of CRC patients. Most identified candidates were involved in either the TNF/FAS signal transduction pathway or transcription factors associated with TP53 or Myc in pathway analysis. A group of 45 colon cancer candidate biomarkers in the 15-65 kDa region of 1D SDS gels was used for follow-up verification and validation in patient plasma samples. After developing multiplexed MRM assays, these candidate biomarkers were quantitated in pools of 25 control and 25 colorectal cancer patient plasma samples. A subset of 22 candidates was selected for subsequent analysis in individual patient specimens. Stable-isotope labeled peptides were used in the final MRM assay to enable absolute quantitation in individual patients and control subjects. Results suggest that several of these biomarkers are superior to CEA and CA-19-9. These biomarkers are identified in Table 1.

Because certain biomarkers identified in Table 1 were found by the inventors to be secreted into serum from the tumor tissue in low abundance, the compositions and methods described herein involve detection and/or quantitative evaluation of these biomarkers, individually or in combination with the other biomarkers in Table 1 and/or with additional known colorectal cancer biomarkers such as the previously identified biomarkers CEA or CA19-9, in a sample, desirably a serum or plasma or blood sample, from a subject. Such detection and evaluation permits diagnosis of colorectal cancer in a less invasive manner than is currently available using a tissue biopsy or a surgical sample. The methods and compositions also may be used as a diagnostic to avoid surgical diagnostic procedures. The identification and use of such a panel of biomarker reagents provides a critical, more precise basis of knowledge to incorporate into pre-clinical and clinical diagnostic assays targeting these biomarkers.

In one embodiment, low-abundance human proteins are present at less than about 100 ng/ml in normal human serum. In other embodiments, low-abundance human proteins are present at less than about 80, 50, 30, 20, 10 or 1 ng/ml in normal human serum. Such difficult-to-detect proteins, which are in lower abundance in the serum, are more tumor-specific than proteins present in greater concentrations in the serum.

Protein abundance levels of biomarkers in blood, in some embodiments, are dependent upon expression levels in tissues of origin (e.g., colorectal tumors), as well as rate of shedding into the blood and rate of clearance from the blood. While increased expression in a tumor often will correlate with increased abundance levels being observed in the blood, this is not necessarily always true. Therefore, the methods and compositions in one aspect refer to compositions that detect protein biomarkers and to protein assay methods. However, one of skill in the art, given the teachings contained herein, would readily understand that nucleic acid expression levels of the biomarkers and reagents and methods for their detections may be similarly practiced, without undue experimentation.

Diagnostic reagents that can detect and measure the target biomarkers and sets of biomarkers identified herein and methods for evaluating the level or ratios of these target biomarkers vs. their level(s) in a variety of reference standards or controls of different conditions or stages in colorectal cancer are valuable tools in the early detection and monitoring of colorectal cancer.

In one embodiment, the compositions and methods allow the detection and measurement of the protein levels or ratios of one or more “target” biomarkers of Table 1 in a biological sample, e.g., a biological fluid. Diagnostic reagents that can detect and measure these target biomarkers and methods for evaluating the level or ratios of these target biomarkers vs. their level(s) in a variety of reference standards or controls of different conditions or stages in colorectal cancer are valuable tools in the early detection and monitoring of colorectal cancer.

### I. DEFINITIONS

“Patient” or “subject” as used herein means a mammalian animal, including a human, a veterinary or farm animal, a domestic animal or pet, and animals normally used for clinical research. In one embodiment, the subject of these methods and compositions is a human.

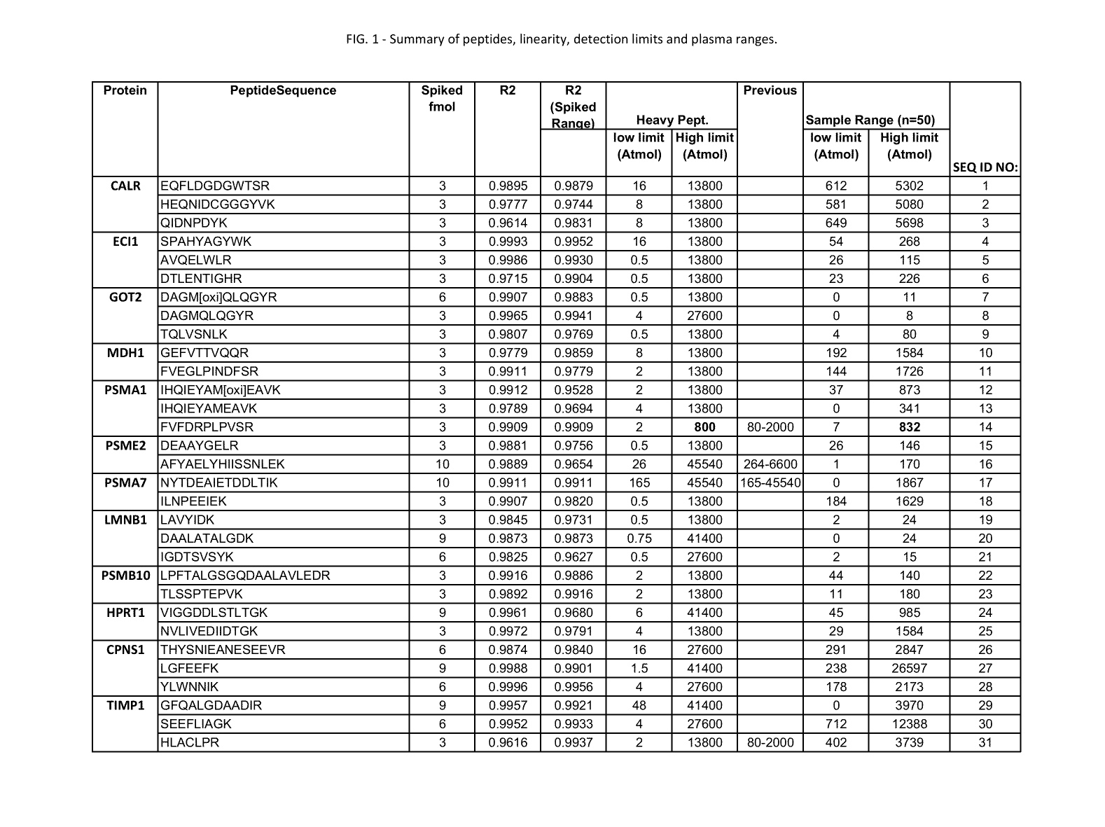

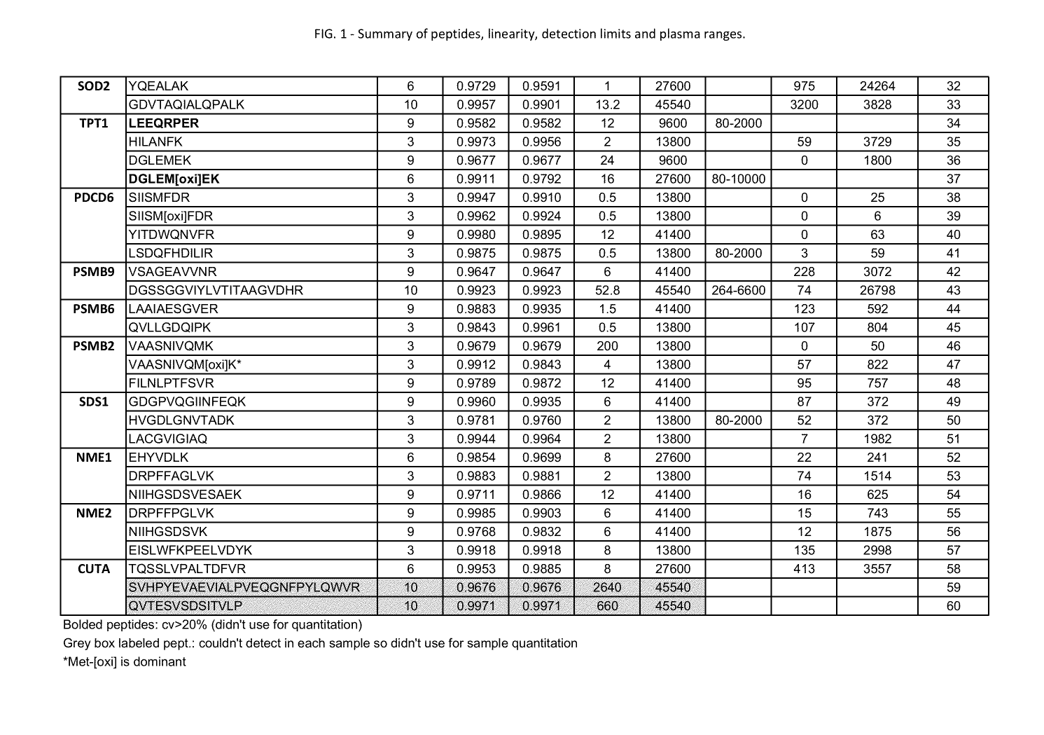

By “biomarker” or “biomarker signature” as used herein is meant a single protein or a combination of proteins or peptide fragments thereof, the protein levels or relative protein levels or ratios of which significantly change (either in an increased or decreased manner) from the level or relative levels present in a subject having one physical condition or disease or disease stage from that of a reference standard representative of another physical condition or disease stage. Throughout this specification, wherever a particular biomarker is identified by name, it should be understood that the term “biomarker” includes those listed in Table 1 below. These biomarkers may be combined to form certain sets of biomarkers or ligands to biomarkers in diagnostic reagents. Still other “additional” biomarkers are mentioned specifically herein in combination with the biomarkers of Table 1. Biomarkers described in this specification include any physiological molecular forms, or modified physiological molecular forms, isoforms, pro-forms, and peptide fragments thereof, unless otherwise specified. It is understood that all molecular forms useful in this context are physiological, e.g., naturally occurring in the species. Preferably the peptide fragments obtained from the biomarkers are unique sequences, such as those exemplified in FIG. 1. However, it is understood that fragments other than those explicitly identified may be obtained readily by one of skill in the art in view of the teachings provided herein.

TABLE 1
SELECTED BIOMARKERS OF COLORECTAL CANCER

| Protein Symbol | Biomarker Protein Name |
|---|---|
| LMNB1 | lamin B1 |
| ECI1 | Enoyl-CoA delta isomerase 1 |
| NME2 | NME/NM23 nucleoside diphosphate kinase 2 |
| NME1 | NME/NM23 nucleoside diphosphate kinase 1 |
| CALR | Calreticulin |
| TPT1 | tumor protein, translationally-controlled 1 |
| PSMB9 | proteasome (prosome, macropain) subunit, beta type 9 |
| PSMB6 | proteasome (prosome, macropain) subunit, beta type 6 |
| PSMA7 | proteasome (prosome, macropain) subunit, alpha type, 7 |
| PSMA1 | proteasome (prosome, macropain) subunit, alpha type, 1 |

In one embodiment, at least one biomarker of Table 1 forms a suitable biomarker signature for use in the methods and compositions. In one embodiment, at least two biomarkers form a suitable biomarker signature for use in the methods and compositions. In another embodiment, at least three biomarkers form a suitable biomarker signature for use in the methods and compositions. In another embodiment, at least four biomarkers form a suitable biomarker signature for use in the methods and compositions. In another embodiment, at least five biomarkers form a suitable biomarker signature for use in the methods and compositions. In another embodiment, at least six biomarkers form a suitable biomarker signature for use in the methods and compositions. In another embodiment, at least seven biomarkers form a suitable biomarker signature for use in the methods and compositions. In another embodiment, at least eight biomarkers form a suitable biomarker signature for use in the methods and compositions. In another embodiment, at least nine biomarkers form a suitable biomarker signature for use in the methods and compositions. In another embodiment, at least ten biomarkers form a suitable biomarker signature for use in the methods and compositions. In still another embodiment, all 10 of the biomarkers of Table 1 can be used alone or with additional biomarkers. Thus, from 1 to 10 of the biomarkers of Table 1, or ligands or reagents that interact with the biomarkers, can be used in diagnostic panels or arrays or kits. In another embodiment, from 1 to 10 of the biomarker/ligands of Table 1 can be used with other known biomarkers for colorectal cancer and their ligands or reagents, so that the biomarker panel or array (or diagnostic reagent or kit) contains at least 2, 3, 4, 5, 6, 7, 8, 9, 10, 11, 12, 15, 20, 30, 40, 50 or at least 60 total biomarkers (or ligands) including any numbers there between. Specific biomarker signatures can include any combination of colorectal cancer biomarkers employing at least one biomarker (or its ligand) from Table 1 and including up to all 10 biomarkers in Table 1, in any combination with another biomarker, such as CEA or CA19-9. Still other biomarkers such as those identified in the references cited herein may also be present in panels with the biomarkers of Table 1.

While these biomarkers may be used individually or in various combinations as signatures, it is contemplated that multiple molecular forms or fragments of each biomarker of Table 1 and fragments/peptides as described in FIG. 1, are similarly useful in the compositions and methods described herein. Other modified molecular forms of the biomarkers include protein modifications such as different glycosylation patterns and other conventional protein modifications of the biomarkers that occur in nature. In one embodiment, multiple molecular forms of the same protein biomarker parallel each other in patient samples. In another embodiment, one molecular form of a biomarker is a better biomarker for a condition than the other. In another embodiment, a ratio of the multiple molecular forms of the same biomarker forms a useful biomarker. Multiple molecular forms of these biomarkers can occur in blood or other biological samples. In certain embodiments, the presence of one or multiple specific molecular forms of the same biomarker, or a ratio of same, rather than overall protein levels is useful as a biomarker for a particular condition specified herein. One skilled in the art may readily reproduce the compositions and methods described herein by use of the sequences of the biomarkers, all of which are publicly available from conventional sources, such as GENBANK, UniProt or NCBI.

By “isoform” or “multiple molecular form” is meant an alternative expression product or variant of a single gene in a given species, including forms generated by alternative splicing, single nucleotide polymorphisms, alternative promoter usage, alternative translation initiation small genetic differences between alleles of the same gene, and posttranslational modifications (PTMs) of these sequences.

By “related proteins” or “proteins of the same family” are meant expression products of different genes or related genes identified as belonging to a common family. Related proteins in the same biomarker family may or may not share related functions. Related proteins can be readily identified as having significant sequence identity either over the entire protein or a significant part of the protein that is typically referred to as a “domain”; typically proteins with at least 20% sequence homology or sequence identity can be readily identified as belonging to the same protein family.

By “homologous protein” is meant an alternative form of a related protein produced from a related gene having a percent sequence similarity or identity of greater than 20%, greater than 30%, greater than 40%, greater than 50%, greater than 60%, greater than 70%, greater than 75%, greater than 80%, greater than 85%, greater than 90%, greater than 95%, greater than 97%, or greater than 99%.

“Reference standard” as used herein refers to the source of the reference biomarker levels. The “reference standard” is preferably provided by using the same assay technique as is used for measurement of the subject's biomarker levels in the reference subject or population, to avoid any error in standardization. The reference standard is, alternatively, a numerical value, a predetermined cutpoint, a mean, an average, a numerical mean or range of numerical means, a numerical pattern, a ratio, a graphical pattern or a protein abundance profile or protein level profile derived from the same biomarker or biomarkers in a reference subject or reference population. In an embodiment, in which expression of nucleic acid sequences encoding the biomarkers is desired to be evaluated, the reference standard can be an expression level of one or more biomarkers or an expression profile.

“Reference subject” or “Reference Population” defines the source of the reference standard. In one embodiment, the reference is a human subject or a population of subjects having no colorectal cancer, i.e., healthy controls or negative controls. In yet another embodiment, the reference is a human subject or population of subjects with one or more clinical indicators of colorectal cancer, but who did not develop colorectal cancer. In still another embodiment, the reference is a human subject or a population of subjects having benign colorectal polyps. In still another embodiment, the reference is a human subject or a population of subjects who had colorectal cancer, following surgical removal of a colorectal tumor. In another embodiment, the reference is a human subject or a population of subjects who had colorectal cancer and were evaluated for biomarker levels prior to surgical removal of a colorectal tumor. Similarly, in another embodiment, the reference is a human subject or a population of subjects evaluated for biomarker levels following therapeutic treatment for colorectal cancer. In still another embodiment, the reference is a human subject or a population of subjects prior to therapeutic treatment for a colorectal cancer. Similarly, in another embodiment, the reference human subject or a population of subjects without colorectal cancer but which tests positive for a protein level of CEA or CA19-9 or other known colorectal cancer biomarker. Similarly, in another embodiment, the reference human subject or a population of subjects with colorectal cancer but which tests negative for a protein level of CEA or CA19-9 or other known colorectal cancer biomarker. In still other embodiments of methods described herein, the reference is obtained from the same test subject who provided a temporally earlier biological sample. That sample can be pre- or post-therapy or pre- or post-surgery.

Other potential reference standards are obtained from a reference that is a human subject or a population of subjects having early stage colorectal cancer. In another embodiment the reference is a human subject or a population of subjects having advanced stage colorectal cancer. In still another embodiment, the reference is a human subject or a population of subjects having a subtype of colorectal cancer. In still another embodiment, the reference is a human subject or a population of subjects having colorectal adenocarcinoma. In still another embodiment, the reference is a human subject or a population of subjects having colorectal carcinoid tumors. In still another embodiment, the reference is a human subject or a population of subjects having gastrointestinal stromal tumors. In still another embodiment, the reference is a subject or a population of subjects having colorectal lymphoma. In another embodiment, the reference is a human subject or a population of subjects having colorectal sarcoma. In another embodiment, the reference standard is a combination of two or more of the above reference standards.

Selection of the particular class of reference standards, reference population, biomarker levels or profiles depends upon the use to which the diagnostic/monitoring methods and compositions are to be put by the physician and the desired result, e.g., initial diagnosis of colorectal cancer or other colorectal condition, clinical management of patients with colorectal cancer after initial diagnosis, including, but not limited to, monitoring for reoccurrence of disease or monitoring remission or progression of the cancer and either before, during or after therapeutic or surgical intervention, selecting among therapeutic protocols for individual patients, monitoring for development of toxicity or other complications of therapy, predicting development of therapeutic resistance, and the like. Such reference standards or controls are the types that are commonly used in similar diagnostic assays for other biomarkers.

“Sample” as used herein means any biological fluid or tissue that contains the colorectal cancer biomarkers of Table 1. The most suitable samples for use in the methods and with the compositions are samples which require minimal invasion for testing, e.g., blood samples, including serum, plasma, whole blood, and circulating tumor cells and fecal matter. It is also anticipated that other biological fluids, such as saliva or urine may be similarly evaluated by the methods described herein. Also, circulating tumor cells or fluids containing them are also suitable samples for evaluation in certain embodiments of this invention. The samples may include biopsy tissue, tumor tissue, surgical tissue, circulating tumor cells, or other tissue. Such samples may further be diluted with saline, buffer or a physiologically acceptable diluent. Alternatively, such samples are concentrated by conventional means. In certain embodiments, e.g., those in which expression levels of nucleic acid sequences encoding the biomarkers are desired to be evaluated, the samples may include biopsy tissue, surgical tissue, circulating tumor cells, or other tissue. In one embodiment, the sample is a tumor secretome, i.e., any fluid or medium containing the proteins secreted from the tumor. These shed proteins may be unassociated, associated with other biological molecules, or enclosed in a lipid membrane such as an exosome. In one embodiment, the sample is plasma. In another embodiment, the sample is fecal matter.

The sample may be provided at any time that is considered biologically relevant to the physician or healthcare provider. In one embodiment, the subject's sample has been provided at a time before any cancer diagnosis. In another embodiment, the subject's sample has been provided at a time after a diagnosis of colorectal cancer. In another embodiment, the subject's sample has been provided at a time following surgical removal of a colorectal tumor or cells. In another embodiment, the subject's sample has been provided at a time prior to surgical removal of a colorectal tumor or cells. In another embodiment, the subject's sample has been provided at a time periodically following therapeutic treatment for colorectal cancer. In another embodiment, the subject's sample has been provided at a time periodically during therapeutic treatment for colorectal cancer. In another embodiment, the subject's sample has been provided at a time following tumor reoccurrence and during treatment or monitoring of the reoccurring tumor. In yet another embodiment, the subject's sample has been provided at a time prior to therapeutic treatment for colorectal cancer. In yet another embodiment, the subject's sample has been provided at a time before diagnosis but where the subject shows clinical symptoms selected from one or more of abdominal pain or abdominal tenderness; blood in the stool; diarrhea, constipation, or other change in bowel habits; narrow stools; or weight loss.

By “significant change in protein level” is meant an increased protein level of a selected biomarker in comparison to that of the selected reference standard or control or relative to a predetermined cutpoint; a decreased protein level of a selected biomarker in comparison to that of the selected reference or control or relative to a predetermined cutpoint; or a combination of a pattern or relative pattern of certain increased and/or decreased biomarkers. In one embodiment, the significant change is an increased protein level.

The degree of change in biomarker protein level can vary with each individual and is subject to variation with each population. For example, in one embodiment, a large change, e.g., 2-3 fold increase or decrease in protein levels of a small number of biomarkers, e.g., from 1 to 5 characteristic biomarkers, is statistically significant. In another embodiment, a smaller relative change in 6 or more (i.e., about 7, 8, 9, 10, 15 or more biomarkers) is statistically significant. The degree of change in any biomarker(s) expression varies with the condition, such as type of colorectal cancer and with the size or spread of the cancer or solid tumor. The degree of change also varies with the immune response of the individual and is subject to variation with each individual. For example, in one embodiment of this invention, a change at or greater than a 1.2 fold increase or decrease in protein level of a biomarker or more than two such biomarkers, or even 3 or more biomarkers, is statistically significant. In another embodiment, a larger change, e.g., at or greater than a 1.5 fold, greater than 1.7 fold or greater than 2.0 fold increase or a decrease in expression of a biomarker(s) is statistically significant. This is particularly true for cancers without solid tumors. Still alternatively, if a single biomarker protein level is significantly increased in biological samples which normally do not contain measurable protein levels of the biomarker, such increase in a single biomarker level may alone be statistically significant. Conversely, if a single biomarker protein level is normally decreased or not significantly measurable in certain biological samples which normally do contain measurable protein levels of the biomarker, such decrease in protein level of a single biomarker may alone be statistically significant.

A change in protein level of a biomarker required for diagnosis or detection by the methods described herein refers to a biomarker whose protein level is increased or decreased in a subject having a condition or suffering from a disease, specifically colorectal cancer, relative to its expression in a reference subject or reference standard. Biomarkers may also be increased or decreased in protein level at different stages of the same disease or condition. The protein levels of specific biomarkers differ between normal subjects and subjects suffering from a disease, benign colorectal polyps, or cancer, or between various stages of the same disease. Protein levels of specific biomarkers differ between pre-surgery and post-surgery patients with colorectal cancer. Such differences in biomarker levels include both quantitative, as well as qualitative, differences in the temporal or relative protein level or abundance patterns among, for example, biological samples of normal and diseased subjects, or among biological samples which have undergone different disease events or disease stages. For the purpose of this invention, a significant change in biomarker protein levels when compared to a reference standard is considered to be present when there is a statistically significant (p<0.05) difference in biomarker protein level between the subject and reference standard or profile, or significantly different relative to a predetermined cut-point.

For example, in one embodiment, the test subject's biomarker(s) levels are compared with a healthy reference standard. If the subject has colorectal cancer, the selected CRC biomarker(s) e.g., those selected from the biomarkers of Table 1, will typically show an increase in protein level from the levels in the healthy reference standard, thus permitting diagnosis of colorectal cancer. In another example, the biomarker(s) of Table 1 differentially change in protein level (either by increased or decreased protein level) when the biomarker levels or relative levels from the sample of a subject having one of the following conditions is compared to a reference subject or population having another of the following physical conditions. These “conditions” include no colorectal cancer, the presence of benign colorectal polyps, the presence of a colorectal cancer, the condition following surgical removal of a colorectal tumor; the condition prior to surgical removal of a colorectal tumor; the condition following a specific therapeutic treatment for a colorectal tumor; the condition prior to a specific therapeutic treatment for a colorectal tumor. It is further anticipated that the biomarker(s) expression levels may change and the changes may be detected during treatment for colorectal cancer. In another embodiment, a condition includes that of a subject having undiagnosed clinical symptoms. Such clinical symptoms may include any of the following: abdominal pain or abdominal tenderness; blood in the stool; diarrhea, constipation, or other change in bowel habits; narrow stools; or weight loss. Still other embodiments of “conditions” as defined above include early stage colorectal cancer; advanced stage colorectal cancer; a subtype of colorectal cancer; colorectal adenocarcinoma; colorectal carcinoid tumors; gastrointestinal stromal tumors; colorectal lymphoma or colorectal sarcoma.

The term “ligand” refers, with regard to protein biomarkers, to a molecule that binds or complexes with a biomarker protein, molecular form or peptide, such as an antibody, antibody mimic or equivalent that binds to or complexes with a biomarker identified herein, a molecular form or fragment thereof. In certain embodiments, in which the biomarker expression is to be evaluated, the ligand can be a nucleotide sequence, e.g., polynucleotide or oligonucleotide, primer or probe.

As used herein, the term “antibody” refers to an intact immunoglobulin having two light and two heavy chains or fragments thereof capable of binding to a biomarker protein or a fragment of a biomarker protein. Thus a single isolated antibody or fragment may be a monoclonal antibody, a synthetic antibody, a recombinant antibody, a chimeric antibody, a humanized antibody, or a human antibody. The term “antibody fragment” refers to less than an intact antibody structure, including, without limitation, an isolated single antibody chain, an Fv construct, a Fab construct, an Fc construct, a light chain variable or complementarity determining region (CDR) sequence, etc.

As used herein, “labels” or “reporter molecules” are chemical or biochemical moieties useful for labeling a ligand, e.g., amino acid, peptide sequence, protein, or antibody. “Labels” and “reporter molecules” include fluorescent agents, chemiluminescent agents, chromogenic agents, quenching agents, radionucleotides, enzymes, substrates, cofactors, inhibitors, radioactive isotopes, magnetic particles, and other moieties known in the art. “Labels” or “reporter molecules” are capable of generating a measurable signal and may be covalently or noncovalently joined to a ligand.

As used herein the term “cancer” refers to or describes the physiological condition in mammals that is typically characterized by unregulated cell growth. More specifically, as used herein, the term “cancer” means any colorectal cancer. In one embodiment, the colorectal cancer is colorectal cancer or subtype as referred to in “conditions” above. In still an alternative embodiment, the cancer is an “early stage” (I or II) colorectal cancer. In still another embodiment, the cancer is a “late stage” (III or IV) colorectal cancer.

The term “tumor,” as used herein, refers to all neoplastic cell growth and proliferation, whether malignant or benign, and all pre-cancerous and cancerous cells and tissues.

By “therapeutic reagent” or “regimen” is meant any type of treatment employed in the treatment of cancers with or without solid tumors, including, without limitation, chemotherapeutic pharmaceuticals, biological response modifiers, radiation, diet, vitamin therapy, hormone therapies, gene therapy, surgical resection, etc.

The term “microarray” refers to an ordered arrangement of binding/complexing array elements or ligands, e.g. antibodies, on a substrate.

In the context of the compositions and methods described herein, reference to “at least two,” “at least five,” etc. of the biomarkers listed in any particular biomarker set means any and all combinations of the biomarkers identified. Specific biomarkers for the biomarker profile do not have to be in rank order in Table 1 but may also include any biomarker, fragment or molecular form, including those described in FIG. 1, as discussed herein.

As used herein, the term “known colorectal cancer biomarker” refers to any biomarker for which an increased or decreased expression level, as compared to a control, has previously been shown to be associated with colorectal cancer. Known colorectal cancer biomarkers include, but are not limited to, CEA and CA19-9.

By “significant change in expression” is meant an upregulation in the expression level of a nucleic acid sequence, e.g., genes or transcript, encoding a selected biomarker, in comparison to the selected reference standard or control; a downregulation in the expression level of a nucleic acid sequence, e.g., genes or transcript, encoding a selected biomarker, in comparison to the selected reference standard or control; or a combination of a pattern or relative pattern of certain upregulated and/or down regulated biomarker genes. The degree of change in biomarker expression can vary with each individual as stated above for protein biomarkers.

The term “polynucleotide,” when used in singular or plural form, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

One skilled in the art may readily reproduce the compositions and methods described herein by use of the amino acid sequences of the biomarkers and other molecular forms, which are publicly available from conventional sources.

It should be understood that while various embodiments in the specification are presented using “comprising” language, under various circumstances, a related embodiment is also be described using “consisting of” or “consisting essentially of” language. It is to be noted that the term “a” or “an”, refers to one or more, for example, “a ligand,” is understood to represent one or more ligands. As such, the terms “a” (or “an”), “one or more,” and “at least one” are used interchangeably herein.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### II. Biomarkers and Biomarker Signatures Useful in the Methods and Compositions

The “targets” of the compositions and methods of these inventions include, in one aspect, biomarkers listed in Table 1, optionally with other biomarkers identified herein, fragments, particularly unique fragments thereof, and molecular forms thereof. In certain embodiments, superior diagnostic tests for diagnosing the existence of colorectal cancer utilize at least one of the ligands that bind or complex with one of biomarkers of Table 1, or one of the fragments or molecular forms thereof. In other embodiments, superior diagnostic tests for diagnosing the existence of or monitoring the progression of CRC utilize multiple ligands, each individually detecting a different specific target biomarker identified herein, or isoform, modified form or peptide thereof. In still other methods, no ligand is necessary, e.g., MRM assays.

In one embodiment the target biomarker of the methods and compositions described herein is lamin B1 (LMNB1). The amino acid sequences for LMNB1 and its molecular forms are publically available, such as in GENBANK. Certain fragments of LMNB1, including those described in FIG. 1, may also be useful as targets in the methods and compositions described herein. It should be understood that, depending upon the context, any reference to LMNB1 herein also refers to any of its peptides or molecular forms.

In one embodiment the target biomarker of the methods and compositions described herein is enoyl-CoA delta isomerase 1 (referred to interchangeably as ECI1, DCI). The amino acid sequence for ECI1 is publically available, such as in GENBANK. Certain fragments of ECI1, including those described in FIG. 1, may also be useful as targets in the methods and compositions described herein. It should be understood that, depending upon the context, any reference to ECI1 herein also refers to any of its peptides or molecular forms.

In one embodiment the target biomarker of the methods and compositions described herein is proteasome (prosome, macropain) subunit, beta type 6 (PSMB6). The amino acid sequence for PSMB6 is publically available, such as in GENBANK. Certain fragments of PSMB6, including those described in FIG. 1, may also be useful as targets in the methods and compositions described herein. It should be understood that, depending upon the context, any reference to PSMB6 herein also refers to any of its peptides or molecular forms.

In one embodiment the target biomarker of the methods and compositions described herein is proteasome (prosome, macropain) subunit, alpha type, 1 (PSMA1). The amino acid sequence for PSMA1 is publically available, such as in GENBANK. Certain fragments of PSMA1, including those described in FIG. 1, may also be useful as targets in the methods and compositions described herein. It should be understood that, depending upon the context, any reference to PSMA1 herein also refers to any of its peptides or molecular forms.

In one embodiment the target biomarker of the methods and compositions described herein is proteasome (prosome, macropain) subunit, beta type 9 (large multifunctional peptidase 2) (PSMB9). The amino acid sequence for PSMB9 is publically available, such as in GENBANK. Certain fragments of PSMB9, including those described in FIG. 1, may also be useful as targets in the methods and compositions described herein. It should be understood that, depending upon the context, any reference to PSMB9 herein also refers to any of its peptides or molecular forms.

In one embodiment the target biomarker of the methods and compositions described herein is NME/NM23 nucleoside diphosphate kinase 2 (NME2). The amino acid sequence for NME2 is publically available, such as in GENBANK. Certain fragments of NME2, including those described in FIG. 1, may also be useful as targets in the methods and compositions described herein. It should be understood that, depending upon the context, any reference to NME2 herein also refers to any of its peptides or molecular forms.

In one embodiment the target biomarker of the methods and compositions described herein is calreticulin (CALR). The amino acid sequence for CALR is publically available, such as in GENBANK. Certain fragments of CALR, including those described in FIG. 1, may also be useful as targets in the methods and compositions described herein. It should be understood that, depending upon the context, any reference to CALR herein also refers to any of its peptides or molecular forms.

In one embodiment the target biomarker of the methods and compositions described herein is tumor protein, translationally-controlled 1 (TPT1). The amino acid sequence for TPT1 is publically available, such as in GENBANK. Certain fragments of TPT1, including those described in FIG. 1, may also be useful as targets in the methods and compositions described herein. It should be understood that, depending upon the context, any reference to TPT1 herein also refers to any of its peptides or molecular forms.

In one embodiment the target biomarker of the methods and compositions described herein is NME/NM23 nucleoside diphosphate kinase 1 (NME1). The amino acid sequence for NME1 is publically available, such as in GENBANK. Certain fragments of NME1, including those described in FIG. 1, may also be useful as targets in the methods and compositions described herein. It should be understood that, depending upon the context, any reference to NME1 herein also refers to any of its peptides or molecular forms.

In one embodiment the target biomarker of the methods and compositions described herein is proteasome (prosome, macropain) subunit, alpha type, 7 (PSMA7). The amino acid sequence for PSMA7 is publically available, such as in GENBANK. Certain fragments of PSMA7, including those described in FIG. 1, may also be useful as targets in the methods and compositions described herein. It should be understood that, depending upon the context, any reference to PSMA7 herein also refers to any of its peptides or molecular forms.

In still another embodiment, the biomarkers targeted by the methods and compositions described herein includes various combinations of these target biomarkers and/or fragments thereof.

In another embodiment, a combination of ligands is used to identify biomarkers including any of the known colorectal cancer biomarkers, Carcinoembryonic Antigen (CEA), Carbohydrate Antigen 19-9 (CA19-9) or tissue inhibitor of metalloproteinases-1 (TIMP-1), or any of their molecular forms, in combination with one or more of the above-noted biomarkers of Table 1.

In one embodiment, the methods and compositions employ ligands that target multiple biomarkers. The multiple biomarker combinations include, without limitation, or consist of, the following exemplary combinations of biomarkers or combinations that include different molecular forms of the same biomarker, for diagnosis of colorectal cancer or for monitoring the progression of the severity of disease or remission of disease:

One or more of LMNB1, ECI1, PSMB6, PSMB9, PSMA1, NME2, CALR, TPT1, NME1, and PSMA7; or

One or more of LMNB1, ECI1, PSMB6, PSMB9, PSMA1, NME2, CALR, TPT1, NME1, and PSMA7 optionally with CEA and/or CA19-9; or

One or both of LMNB1 and ECI1; or

One or more of LMNB1, ECI1, and NME2; or

One or more of LMNB1, ECI1, NME2, and CALR; or

One or more of LMNB1, ECI1, NME2, CALR, and NME1; or

One or more of LMNB1, ECI1, NME2, CALR, NME1, and TPT1; or

One or more of LMNB1, ECI1, NME2, CALR, NME1, and TPT1 optionally with CEA; or

One or more of LMNB1, ECI1, NME2, CALR, NME1, and TPT1 optionally with CA19-9; or

LMNB1 or ECI1 with one or more of NME2, CALR, NME1, and TPT1; or

LMNB1 or ECI1 with one or more of NME2, CALR, NME1, and TPT1 optionally with CEA; or

LMNB1 or ECI1 with one or more of NME2, CALR, NME1, and TPT1 optionally with CA19-9; or

One or more of LMNB1, ECI1, NME2, CALR, NME1, and TPT1 with one or more of PSMB6, PSMB9, PSMA1, and PSMA7; or

One or more of LMNB1, ECI1, NME2, CALR, NME1, and TPT1 with one or more of PSMB6, PSMB9, PSMA1, and PSMA7 and optionally with CEA and/or CA19-9; or

Still other combinations of the Table 1 biomarkers can be targeted in combination with other known colorectal cancer biomarkers to produce a desired signature for one of the colorectal cancer-related conditions described above.

For example, among desirable biomarker signatures are signatures that comprise, or consist of, at least 2, 3, 4, 5, 6, 7, 8, 9, 10, or all 10 of the biomarkers of Table 1, including optionally CEA and/or CA19-9 or any other known colorectal cancer biomarker or molecular forms or peptides thereof.

As further stated above, the biomarkers/biomarker signatures described above, can in another embodiment, refer to nucleic acid sequences, genes and transcripts encoding the biomarkers and expression profiles thereof.

### III. Diagnostic Reagents, Devices and Kits

### A. Labeled or Immobilized Biomarkers or Peptides or Molecular Forms

In one embodiment, diagnostic reagents or devices for use in the methods of diagnosing colorectal cancer include one or more target biomarker or peptide fragment identified in Table 1 or FIG. 1 herein, or molecular forms thereof, associated with a detectable label or portion of a detectable label system. In another embodiment, a diagnostic reagent includes one or more target biomarker or peptide fragment thereof identified in Table 1, immobilized on a substrate. In still another embodiment, combinations of such labeled or immobilized biomarkers are suitable reagents and components of a diagnostic kit or device.

In another aspect, suitable embodiments of such labeled or immobilized reagents include at least one, 2, 3, 4, 5, 6, 7, 8, 9, 10, or all 10 of biomarkers of Table 1 or their unique peptide fragments therein.

In one aspect the reagent, device or kit comprises or consists of ligands that individually specifically complex with, bind to, or quantitatively detect or identify multiple isoforms of any of biomarkers LMNB1, ECI1, NME2, CALR, NME1, TPT1, PSMB6, PSMB9, PSMA1, and PSMA7. In one embodiment, the ligands are selected from those listed in FIG. 1.

In another aspect, the reagent, device or kit comprises or consists of ligands that individually specifically complex with, bind to, or quantitatively detect or identify two or more biomarkers selected from any of the specific combinations of biomarkers recited above.

In another embodiment suitable embodiments include at least one biomarker, CEA, or an isoform, pro-form, modified molecular form, or peptide fragment thereof.

In another embodiment suitable embodiments include at least one biomarker, CA19-9, or an isoform, pro-form, modified molecular form, or peptide fragment thereof.

Any combination of labeled or immobilized biomarkers can be assembled in a diagnostic kit or device for the purposes of diagnosing colorectal cancer, such as those combinations of biomarkers discussed herein.

For these reagents, the labels may be selected from among many known diagnostic labels, including those described above. Similarly, the substrates for immobilization in a device may be any of the common substrates, glass, plastic, a microarray, a microfluidics card, a chip, a bead or a chamber.

### B. Labeled or Immobilized Ligands that Bind or Complex with the Biomarkers

In another embodiment, the diagnostic reagent or device includes a ligand that binds to or complexes with a biomarker of Table 1 or a unique peptide thereof including those described in FIG. 1, or a molecular form thereof or a combination of such ligands.

In one embodiment, such a ligand desirably binds to a protein biomarker or a unique peptide contained therein, and can be an antibody which specifically binds a single biomarker of Table 1, or a unique peptide in that single biomarker including those described in FIG. 1. Various forms of antibody, e.g., polyclonal, monoclonal, recombinant, chimeric, as well as fragments and components (e.g., CDRs, single chain variable regions, etc.) or antibody mimics or equivalents may be used in place of antibodies. The ligand itself may be labeled or immobilized.

In another embodiment, suitable labeled or immobilized reagents include at least 2, 3, 4, 5, 6, 7 8, 9, 10 or 11 or more ligands. Each ligand binds to or complexes with a single biomarker protein/peptide, fragment, or molecular form of the biomarker(s) of Table 1. In some of the embodiments in which the combination of biomarkers includes CEA and/or CA19-9 or another known additional biomarker that may be in higher abundance in serum, ligands to each of these additional biomarkers may be employed in the diagnostic reagent.

Any combination of labeled or immobilized biomarker ligands can be assembled in a diagnostic kit or device for the purposes of diagnosing colorectal cancer or monitoring the progress of colorectal cancer.

Thus, a kit or device can contain multiple reagents or one or more individual reagents. For example, one embodiment of a composition includes a substrate upon which the biomarkers or ligands are immobilized. In another embodiment, the kit also contains optional detectable labels, immobilization substrates, optional substrates for enzymatic labels, as well as other laboratory items. In one embodiment, the reagents are designed for use in plasma.

The diagnostic reagents, devices, or kits compositions based on the biomarkers of Tables 1 or fragments thereof described herein, optionally associated with detectable labels, can be presented in the format of a microfluidics card, a chip or chamber, a bead or a kit adapted for use with assays formats such as sandwich ELISAs, multiple protein assays, platform multiplex ELISAs, such as the BioRad Luminex platform, Mass spectrometry quantitative assays, or PCR, RT-PCR or Q PCR techniques.

In one embodiment, a kit includes multiple antibodies directed to bind to one or more of the combinations of biomarkers described above, wherein the antibodies are associated with detectable labels.

In another embodiment, the reagent ligands are nucleotide sequences, the diagnostic reagent is a polynucleotide or oligonucleotide sequence that hybridizes to gene, gene fragment, gene transcript or nucleotide sequence encoding a biomarker of Table 1 or encoding a unique peptide thereof including those described in FIG. 1. Such a polynucleotide/oligonucleotide can be a probe or primer, and may itself be labeled or immobilized. In one embodiment, ligand-hybridizing polynucleotide or oligonucleotide reagent(s) are part of a primer-probe set, and the kit comprises both primer and probe. Each said primer-probe set amplifies a different gene, gene fragment or gene expression product that encodes a different biomarker of Table 1, optionally including one or more additional known biomarkers, such as CEA or CA19-9. For use in the compositions the PCR primers and probes are preferably designed based upon intron sequences present in the biomarker gene(s) to be amplified selected from the gene expression profile. The design of the primer and probe sequences is within the skill of the art once the particular gene target is selected. The particular methods selected for the primer and probe design and the particular primer and probe sequences are not limiting features of these compositions. A ready explanation of primer and probe design techniques available to those of skill in the art is summarized in U.S. Pat. No. 7,081,340, with reference to publically available tools such as DNA BLAST software, the Repeat Masker program (Baylor College of Medicine), Primer Express (Applied Biosystems); MGB assay-by-design (Applied Biosystems); Primer3 (Steve Rozen and Helen J. Skaletsky (2000) Primer3 on the WWW for general users and for biologist programmers and other publications.

In general, optimal PCR primers and probes used in the compositions described herein are generally 17-30 bases in length, and contain about 20-80%, such as, for example, about 50-60% G+C bases. Melting temperatures of between 50 and 80° C., e.g. about 50 to 70° C. are typically preferred.

The selection of the ligands, biomarker sequences, their length, suitable labels and substrates used in the reagents and kits are routine determinations made by one of skill in the art in view of the teachings herein of which biomarkers form a signature suitable for the diagnosis of colorectal cancer.

### IV. Methods for Diagnosing or Monitoring Colorectal Cancer

In another embodiment, a method for diagnosing or detecting or monitoring the progress of colorectal cancer in a subject comprises, or consists of, a variety of steps.

A. Sample Preparation

The test sample is obtained from a human subject who is to undergo the testing or treatment. The subject's sample can in one embodiment be provided before initial diagnosis, so that the method is performed to diagnose the existence of a colorectal cancer. In another embodiment, depending upon the reference standard and markers used, the method is performed to diagnose the stage of colorectal cancer. In another embodiment, depending upon the reference standard and markers used, the method is performed to diagnose the type or subtype of colorectal cancer from the types and subtypes identified above. In another embodiment, the subject's sample can be provided after a diagnosis, so that the method is performed to monitor progression of a colorectal cancer. In another embodiment, the sample can be provided prior to surgical removal of a colorectal tumor or prior to therapeutic treatment of a diagnosed colorectal cancer and the method used to thereafter monitor the effect of the treatment or surgery, and to check for relapse. In another embodiment, the sample can be provided following surgical removal of a colorectal tumor or following therapeutic treatment of a diagnosed colorectal cancer, and the method performed to ascertain efficacy of treatment or relapse. In yet another embodiment the sample may be obtained from the subject periodically during therapeutic treatment for a colorectal cancer, and the method employed to track efficacy of therapy or relapse. In yet another embodiment the sample may be obtained from the subject periodically during therapeutic treatment to enable the physician to change therapies or adjust dosages. In one or more of these embodiments, the subject's own prior sample can be employed in the method as the reference standard.

Preferably where the sample is a fluid, e.g., blood, serum or plasma, obtaining the sample involves simply withdrawing and preparing the sample in the traditional fashion for contact with the diagnostic reagent. Where the sample is a tissue or tumor sample, it may be prepared in the conventional manner for contact with the diagnostic reagent. Where the sample is a tumor secretome, the sample may be prepared as described in the examples below. Where the sample is fecal matter (stool), the sample may be prepared as conventional in the art, e.g., as described in US Patent Publication No. US 2011/0129860, which is incorporated herein by reference.

The method further involves contacting the sample obtained from a test subject with a diagnostic reagent as described above under conditions that permit the reagent to bind to or complex with one or more biomarker(s) of Table 1 which may be present in the sample. This method may employ any of the suitable diagnostic reagents or kits or compositions described above.

B. Measuring Biomarker Levels

Thereafter, a suitable assay is employed to detect or measure in the sample the protein level (actual or relative) of one or more biomarker(s) of Table 1. Alternatively, a suitable assay is employed to generate a protein abundance profile (actual or relative or ratios thereof) of multiple biomarkers of Table 1 from the sample or of multiple different molecular forms of the same biomarker or both. In another embodiment, the above method further includes measuring in the biological sample of the subject the protein level of an additional biomarker, such as CEA, CA19-9 or other known colorectal cancer biomarker. In another embodiment, the above method further includes measuring in the biological sample of the subject the protein levels of two or more additional biomarkers which form a biomarker protein abundance signature for colorectal cancer.

The measurement of the biomarker(s) in the biological sample may employ any suitable ligand, e.g., antibody, antibody mimic or equivalent (or antibody to any second biomarker) to detect the biomarker protein. Such antibodies may be presently extant in the art or presently used commercially, such as those available as part of commercial antibody sandwich ELISA assay kits or that may be developed by techniques now common in the field of immunology. A recombinant molecule bearing the binding portion of a biomarker antibody, e.g., carrying one or more variable chain CDR sequences that bind e.g., LMNB1, ECI1, NME2, CALR, NME1, TPT1, PSMB6, PSMB9, PSMA1, and PSMA7, etc. may also be used in a diagnostic assay. As used herein, the term “antibody” may also refer, where appropriate, to a mixture of different antibodies or antibody fragments that bind to the selected biomarker. Such different antibodies may bind to different biomarkers or different portions of the same biomarker protein than the other antibodies in the mixture. Such differences in antibodies used in the assay may be reflected in the CDR sequences of the variable regions of the antibodies. Such differences may also be generated by the antibody backbone, for example, if the antibody itself is a non-human antibody containing a human CDR sequence, or a chimeric antibody or some other recombinant antibody fragment containing sequences from a non-human source. Antibodies or fragments useful in the method may be generated synthetically or recombinantly, using conventional techniques or may be isolated and purified from plasma or further manipulated to increase the binding affinity thereof. It should be understood that any antibody, antibody fragment, or mixture thereof that binds one of the biomarkers of Table 1 or a particular sequence of the selected biomarker e.g., as defined in FIG. 1 may be employed in the methods described herein, regardless of how the antibody or mixture of antibodies was generated.

Similarly, the antibodies may be tagged or labeled with reagents capable of providing a detectable signal, depending upon the assay format employed. Such labels are capable, alone or in concert with other compositions or compounds, of providing a detectable signal. Where more than one antibody is employed in a diagnostic method, e.g., such as in a sandwich ELISA, the labels are desirably interactive to produce a detectable signal. Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods and devices of this invention are detectable by other means, e.g., colored latex microparticles (Bangs Laboratories, Indiana) in which a dye is embedded may be used in place of enzymes to provide a visual signal indicative of the presence of the resulting selected biomarker-antibody complex in applicable assays. Still other labels include fluorescent compounds, radioactive compounds or elements. Preferably, an anti-biomarker antibody is associated with, or conjugated to a fluorescent detectable fluorochrome, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

Detectable labels for attachment to antibodies useful in diagnostic assays and devices of this invention may be easily selected from among numerous compositions known and readily available to one skilled in the art of diagnostic assays. The biomarker-antibodies or fragments useful in this invention are not limited by the particular detectable label or label system employed. Thus, selection and/or generation of suitable biomarker antibodies with optional labels for use in this invention is within the skill of the art, provided with this specification, the documents incorporated herein, and the conventional teachings of immunology.

Similarly the particular assay format used to measure the selected biomarker in a biological sample may be selected from among a wide range of protein assays, such as described in the examples below. Suitable assays include enzyme-linked immunoassays, sandwich immunoassays (ELISA), homogeneous assays, immunohistochemistry formats, or other conventional assay formats. In one embodiment, a serum/plasma sandwich ELISA is employed in the method. In another embodiment, a mass spectrometry-based assay is employed. In another embodiment, a MRM assay is employed, in which antibodies are used to enrich the biomarker in a manner analogous to the capture antibody in sandwich ELISAs.

One of skill in the art may readily select from any number of conventional immunoassay formats to perform this invention.

Other reagents for the detection of protein in biological samples, such as peptide mimetics, synthetic chemical compounds capable of detecting the selected biomarker may be used in other assay formats for the quantitative detection of biomarker protein in biological samples, such as high pressure liquid chromatography (HPLC), immunohistochemistry, etc.

Employing ligand binding to the biomarker proteins or multiple biomarkers forming the signature enables more precise quantitative assays, as illustrated by the multiple reaction monitoring (MRM) mass spectrometry (MS) assays. As an alternative to specific peptide-based MRM-MS assays that can distinguish specific protein isoforms and proteolytic fragments, the knowledge of specific molecular forms of biomarkers allows more accurate antibody-based assays, such as sandwich ELISA assays or their equivalent. Frequently, the isoform specificity and the protein domain specificity of immune reagents used in pre-clinical (and some clinical) diagnostic tests are not well defined.

In one embodiment, suitable assays for use in these methods include immunoassays using antibodies or ligands to the above-identified biomarkers and biomarker signatures. In another embodiment, a suitable assay includes a multiplexed MRM based assay for two more biomarkers that include one or more of the proteins/unique peptides in Table 1 or FIG. 1. It is anticipated that ultimately the platform most likely to be used in clinical assays will be multi-plexed or parallel sandwich ELISA assays or their equivalent, primarily because this platform is the technology most commonly used to quantify blood proteins in clinical laboratories. MRM-MS assays may continue to be used productively to help evaluate the isoform/molecular form specificity of any existing immunoassays or those developed in the future.

### C. Detection of a Change in Biomarker Abundance Level and Diagnosis

The measurement of the protein level of the one or more biomarker(s) in the subject's sample or the protein abundance profile of multiple said biomarkers as detected by the use of the assays described above allows for comparison with the level of the same biomarker or biomarkers in a reference standard or reference profile. In one embodiment, the comparing step of the method is performed by a computer processor or computer-programmed instrument that generates numerical or graphical data useful in the appropriate diagnosis of the condition. Optionally, the comparison may be performed manually.

The detection or observation of a change in the protein level of a biomarker or biomarkers in the subject's sample from the same biomarker or biomarkers in the reference standard can indicate an appropriate diagnosis. An appropriate diagnosis can be identifying a risk of developing colorectal cancer, a diagnosis of colorectal cancer (or stage or type thereof), a diagnosis or detection of the status of progression or remission of colorectal cancer in the subject following therapy or surgery, a determination of the need for a change in therapy or dosage of therapeutic agent. The method is thus useful for early diagnosis of disease, for monitoring response or relapse after initial diagnosis and treatment or to predict clinical outcome or determine the best clinical treatment for the subject.

In one embodiment, the change in protein level of each biomarker can involve an increase of a biomarker or multiple biomarkers in comparison to the specific reference standard. In one embodiment, a selection or all of the biomarkers of Table 1 are increased in a subject sample from a patient having colorectal cancer when compared to the levels of these biomarkers from a healthy reference standard. In another embodiment, a selection or all of the biomarkers of Table 1 are increased in a subject sample from a patient having colorectal cancer prior to therapy or surgery, when compared to the levels of these biomarkers from a post-surgery or post-therapy reference standard.

In another embodiment, the change in protein level of each biomarker can involve a decrease of a biomarker or multiple biomarkers in comparison to the specific reference standard. In one embodiment, a selection or all of the biomarkers of Table 1 are decreased in a subject sample from a patient having colorectal cancer following surgical removal of a tumor or following chemotherapy/radiation when compared to the levels of these biomarkers from a pre-surgery/pre-therapy colorectal cancer reference standard or a reference standard which is a sample obtained from the same subject pre-surgery or pre-therapy.

In still other embodiments, the changes in protein levels of the biomarkers may be altered in characteristic ways if the reference standard is a particular type of colorectal cancer, e.g., adenocarcinoma, carcinoid tumor, gastrointestinal stromal tumor, lymphoma or sarcoma, or if the reference standard is derived from benign colorectal cysts or polyps.

The results of the methods and use of the compositions described herein may be used in conjunction with clinical risk factors to help physicians make more accurate decisions about how to manage patients with colorectal cancers. Another advantage of these methods and compositions is that diagnosis may occur earlier than with more invasive diagnostic measures.

### D. Alternative Embodiments

In an alternative embodiment, the method of diagnosis or risk of diagnosis involves using the nucleic acid hybridizing reagent ligands described above to detect a significant change in expression level of the subject's sample biomarker or biomarkers from that in a reference standard or reference expression profile which indicates a diagnosis, risk, or the status of progression or remission of colorectal cancer in the subject. These methods may be performed in other biological samples, e.g., biopsy tissue samples, tissue removed by surgery, or tumor cell samples, including circulating tumor cells isolated from the blood, to detect or analyze a risk of developing a colorectal cancer, as well as a diagnosis of same. Such methods are also known in the art and include contacting a sample obtained from a test subject with a diagnostic reagent comprising a ligand which is a nucleotide sequence capable of hybridizing to a nucleic acid sequence encoding a biomarker of Table 1, said ligand associated with a detectable label or with a substrate. Thereafter one would detect or measure in the sample or from an expression profile generated from the sample, the expression levels of one or more of the biomarkers or ratios thereof. The expression level(s) of the biomarker(s) in the subject's sample or from an expression profile or ratio of multiple said biomarkers are then compared with the expression level of the same biomarker or biomarkers in a reference standard. A significant change in expression level of the subject's sample biomarker or biomarkers from that in the reference standard indicates a diagnosis, risk, or the status of progression or remission of colorectal cancer in the subject.

Suitable assay methods include methods based on hybridization analysis of polynucleotides, methods based on sequencing of polynucleotides, proteomics-based methods or immunochemistry techniques. The most commonly used methods known in the art for the quantification of mRNA expression in a sample include northern blotting and in situ hybridization; RNAse protection assays; and PCR-based methods, such as reverse transcription PCR (RT-PCR) or quantitative PCR (qPCR). Alternatively, antibodies may be employed that can recognize specific DNA-protein duplexes. The methods described herein are not limited by the particular techniques selected to perform them. Exemplary commercial products for generation of reagents or performance of assays include TRI-REAGENT, Qiagen RNeasy mini-columns, MASTERPURE Complete DNA and RNA Purification Kit (EPICENTRE®, Madison, Wis.), Paraffin Block RNA Isolation Kit (Ambion, Inc.) and RNA Stat-60 (Tel-Test), the MassARRAY-based method (Sequenom, Inc., San Diego, Calif.), differential display, amplified fragment length polymorphism (iAFLP), and BeadArray™ technology (Illumina, San Diego, Calif.) using the commercially available Luminex100 LabMAP system and multiple color-coded microspheres (Luminex Corp., Austin, Tex.) and high coverage expression profiling (HiCEP) analysis.

The comparison of the quantitative or relative expression levels of the biomarkers may be done analogously to that described above for the comparison of protein levels of biomarkers.

### E. Non-Ligand-Based Analysis

In another aspect, a method for diagnosing or detecting or monitoring the progress of colorectal cancer in a subject involves non-ligand based methods, such as mass spectrometry. For example, proteins in a biological sample obtained from a test subject may be contacted with a chemical or enzymatic agent and the proteins, including the biomarkers contained therein fragmented in the sample. The digested sample or portions thereof are injected into a mass spectrometer and the protein levels or ratios of one or more of the biomarkers of Table 1, optionally with other known biomarkers, modified molecular forms, peptides and unique peptides or ratios thereof, are quantitatively identified or measured by mass spectrometry. The protein levels of the biomarkers in the subject's sample are then compared with the level of the same biomarker or biomarkers in a reference standard or to a predetermined cutoff derived from the reference standard. In one embodiment, the agent is a proteolytic enzyme. In another embodiment, the agent is trypsin.

In one embodiment, a significant change in protein level of the subject's sample biomarker or biomarkers from that in the reference standard or from a predetermined cutoff indicates a diagnosis, risk, or the status of progression or remission of colorectal cancer in the subject.

Thus, the various methods, devices and steps described above can be utilized in an initial diagnosis of colorectal cancer or other colorectal condition, as well as in clinical management of patients with colorectal cancer after initial diagnosis. Uses in clinical management of the various devices, reagents and assay methods, include without limitation, monitoring for reoccurrence of disease or monitoring remission or progression of the cancer and either before, during or after therapeutic or surgical intervention, selecting among therapeutic protocols for individual patients, monitoring for development of toxicity or other complications of therapy, and predicting development of therapeutic resistance.

In one embodiment, the method involves enriching the biomarker protein or one or more peptides produced by specific proteolysis in the sample by contacting the sample with an antibody prior to injecting into a mass spectrometer in a manner analogous to a capture antibody in a conventional sandwich ELISA. In another embodiment, the method involves depleting the sample of non-target proteins prior to injecting sample into a mass spectrometer. The depletion may also be performed using antibodies to the non-targets. The method described herein may use liquid chromatographic mass spectrometry, such as HPLC. One such method is described in detail in the Examples below.

### V. EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein.

### Example 1

### Materials and Methods

A. Materials

Medium and Insulin were obtained from Sigma Aldrich (St. Louis, Mo.) and L-glutamine, penicillin, streptomycin, transferring and NaHCO3 were came from Gibco® Invitrogen (Carlsbad, Calif.). Mouse EGF was purchased from BD Biosciences (San Jose, Calif.). Ultra pure urea, thiourea, DTT, and CHAPS were from GE Healthcare, Ltd., (Giles, U.K.) The Bradford protein assay kit was from Thermo Fisher Scientific (Waltham, Mass.) and sequencing-grade porcine trypsin was from Promega Corporation (Madison, Wis.). NuPAGE precast gels were obtained from Invitrogen Corporation (Carlsbad, Calif.). Trypsin digest was performed with porcine sequencing grade modified trypsin (Promega, Madison, Wis.). HPLC-grade ACN was obtained from J. T. Baker (Phillipsburg, N.J.).

B. Cell Culture and Secretome

Human colorectal cancer cells (WC013) were obtained from our collaborator Dr. Dorothee Herlyn at the Wistar Institute. These cells were established from a 47-year-old male patient with primary adenocarcinoma of stage C. Cells were maintained in vitro in CRC medium, which contains MCDB 201 medium supplemented with 20% L15 medium, 20% heat-inactivated FBS (fetal bovine serum), 5 mM L-glutamine, 0.05% NaHCO₃, 100 U/ml penicillin, 50 ug/ml streptomycin, 2 ug/ml of Bovine Insulin, 2 ug/ml of human transferrin, and 5 ng/ml of mouse EGF. Cells were cultured at 37° C. in a humidified incubator in an atmosphere of 5% CO2 in air, as described previously (Jacob, Int J Cancer, 1997). Cell viability was assessed by trypan blue dye exclusion using Countess™ automated cell counter (Invitrogen). When cells were at 85% confluence in 100 mm culture dishes, cells of 3 individual 100 mm dishes were washed three times with CRC medium excluding FBS. Cells were incubated with 4 ml of CRC medium without FBS for 16 h to get the secretome of WC013. About 12 ml of secretome was collected, filtered using 0.22 mm filter (Millipore) at 1000×g for 5 min, and centrifuged at 100,000×g for 60 min. Supernatant of 100,000 g was concentrated at 2000×g for 40 min using 10K MWCO spin concentrator (Millipore) until volume was reached to 50 ul.

C. Colorectal Cancer Growth In Vivo.

Animal studies were approved by the Wistar Institute's Animal Research Committee of the Philadelphia, in accordance with the Institutional Animal Care and Use Committee. Xenograft mice, derived from Wistar CB17 Severe Combined Immunodeficiency Disease (SCID) mice, were kept in pathogen-free conditions and used at 6 to 8 weeks of age. SCID mice were housed five per cage in a room with controlled temperature (20-22° C.). 10 female SCID mice of 7 weeks old were injected 75 μL of human adenocarcinoma WC013 (2.5×10⁶ cells/spot, total 2 spots/mouse) mixed 50:50 with Matrigel (BD, Franklin Lakes, N.J.) subcutaneously on the dorsal flank. Tumors were measured once per week with calipers, and tumor volumes were calculated based on width×length×depth. After 7 or 8 weeks when tumor size was reached approximately 1000 mm³, the animals were anesthetized, and whole blood was taken by cardiac puncture using a 1 mL with 23 G ½ needle syringe. Care was taken to minimize hemolysis during blood extraction. Blood was collected into EDTA tubes and centrifuged at 2000×g for 10 min at room temperature to obtain plasma and frozen/stored at −80° C. The tumor including some organs (e.g. liver, heart, kidney, spleen, and lung) were preserved as freshly frozen and a small piece of tumor was kept in 10% formalin for hematoxylin/eosin staining of paraffin embedded sections to inspect the necrosis of tumor.

D. Major Mouse Plasma Protein Depletion

Removal of the three most abundant proteins in mouse plasma was achieved as reported previously (Tang, H. Y. et al, A xenograft mouse model coupled with in-depth plasma proteome analysis facilitates identification of novel serum biomarkers for human ovarian cancer. J Proteome Res 2012, 11, (2), 678-91). Briefly a total of 640 μl pooled plasma (24.6 mg) was applied to the antibody column in fourteen serial injections. For all experiments, the flow-through fractions from sequential injections were collected, pooled and concentrated to 400 μl using a 5K MWCO spin concentrator (Millipore Corporation, Bedford, Mass.). Affinity-bound major proteins were eluted with the manufacturer's elution buffer, neutralized with 1 M NaOH, concentrated to 1 mL, and stored at −20° C.

For 3-D experiment, 90 μl of concentrated unbound fraction (depleted plasma) was reduced with 10 mM DTT for 1 h at 37° C. in 20 mM Tris-Cl buffer, pH 8.0. The mixture was alkylated with 60 mM iodoacetamide (IMA) for another 1 h at 37° C. Reaction was quenched by adding DTT to 1% (64.8 mM) final concentration. Fifty mM N, N-dimethylacrylamide (DMA) was used for alkylation of depleted plasma in the 4-D method.

E. MicroSol-IEF Fractionation for the 4D Experiment

Prior to MicroSol-IEF, 2.7 mg (150 μL) of depleted mouse plasma proteins were treated as previously reported (Tang et al, 2012 cited above). Reaction was quenched with DTT at 1% final concentration, followed by addition of 2 M thiourea, 4% CHAPS, 1% pH 3-7 ZOOM focusing buffer, and 1% of 7-12 pH ZOOM focusing buffer. MicroSol-IEF was performed using a ZOOM IEF Fractionator (Invitrogen, Carlsbad, Calif.) configured as a four-chamber device, with pH 3.0, 4.6, 5.4, 6.2, and 12.0 membranes. The alkylated sample was loaded onto the pH 5.4-6.2 separation chamber and separation was performed as previously reported (Zuo, Proteomics, 2002). Other settings for this experiment were the same as previously described (Wang, H., Chang-Wong, T., Tang, H-Y., and Speicher, D. W. 2010. Comparison of extensive protein fractionation and repetitive LC-MS/MS analyses on depth of analysis for complex proteomes. J. Proteome Res. 9:1032-40, which is incorporated by reference herein).

F. 1-D SDS-PAGE/Trypsin Digestion

Each experiment was initially analytically separated on 10% NuPAGE minigels (with MOPS running buffer) using standard separation conditions. Preparative SDS-PAGE (12% gel with MES buffer) was separately run using secretome, depleted mouse plasma, the four MicroSol IEF fractions and five MicroSol IEF membrane extracts in an analogous manner to the analytical run, except the separation distance was limited as following: 22 mm for 2-D, 43 mm for 3-D, and 34 mm or 12 mm for 4-D method to minimize required numbers of fractions and gel volume per fraction. The highest possible protein amounts that did not cause extensive band distortion were loaded. Proteins were visualized by staining with Colloidal Blue (Invitrogen, Carlsbad, Calif.). For maximum loading of proteins onto lanes of gel, each fraction and membrane extraction of 4-D method was concentrated using ethanol precipitation overnight at 0° C. After centrifugation of protein-ethanol mixture for 30 min at 1500 g at 4° C., the pellets were reconstructed with 20 mM Tris (pH 8.5) containing 1% SDS and 1% 2-ME. Trypsin digestion was performed as previously described (Wang, H., Chang-Wong, T., Tang, H-Y., and Speicher, D. W. 2010. Comparison of extensive protein fractionation and repetitive LC-MS/MS analyses on depth of analysis for complex proteomes. J. Proteome Res. 9:1032-40. PMID: 20014860. PMCID: PMC2870931, which is incorporated by reference herein.)

Typically four lanes of gel, except 2 lanes in 2-D secretome analysis, were loaded with following volume in each method; 40 μL of concentrated secretome for 2-D, 32 μL of depleted mouse plasma for 3-D method, 104 μL of fraction for 4-D_{—}85 run and 98 μL of fractions or 230 μL of membrane extractions for 4-D_{—}175 analysis. Each lane was sliced into 4 mm×1 mm×1 mm thick gel pieces (pixel), two adjacent pieces were combined in a digestion well and were digested overnight with trypsin. The 2-D secretome experiment consisted of a biological and technical duplicate of 44 digestions (total 88 pixels). Eighty six digestions for the 3-D method were composed of replicates 43 digestions (3D_{—}83). The 4D_{—}85 analysis utilized four fractions in which the digestions were combined with corresponding membrane extraction. There were 62 digestions for fraction 2 and 3, and 23 digestions for fraction 1 and 4, totaling 85 digestions. Another 4-D 175 method was performed with four fractions and five membrane extractions. One hundred one digestions for fraction 2, 3, and 4, and seventy four digestions for fraction 1 and membrane extractions, total 175 digestions (4D_{—}175). (See, Tang, H.-Y., Beer, L., Chang-Wong, T., Hammond, R., Gimotty, P., Coukos, G., and Speicher, D. W. 2012. A xenograft mouse model coupled with in-depth plasma proteome analysis facilitates identification of novel serum biomarkers for human ovarian cancer. J Proteome Res. 11:678-91, which is incorporated by reference herein.)

G. LC-MS/MS

In-gel trypsin digestions were injected into a 75 μm i.d.×25 cm PicoFrit (New Objective, Inc., Woburn, Mass.) in-house column packed with 3 μm Magic C18 resin and peptides were separated by nano-Aquity UPLC system (Waters, Milford, Mass.) interfaced with a LTQ-OribiTrap mass spectrometer (Thermo Fisher Scientific, Waltham, Mass.). For each analysis, 8 μL of trypsin digest was loaded onto the column using solvent A (0.1% formic acid in Milli-Q [Millipore Corporation, Billerica, Mass.] water). Peptides were subsequently eluted using the following gradient conditions with 0.1% formic acid in acetonitrile as solvent B: 1-28% B over 42 min, 28-50% B over 25.5 min, 50-80% B over 5 min, and 80% B for 5 min. To minimize carryover, a 25 min blank injection was run between each sample. Forty three 3-D samples were analyzed first, followed by another replicated injection of the same 3-D samples. Then, 4D_{—}85 samples were analyzed, followed by 4D_{—}175 samples in LC-MS/MS. Lastly, 88 secretome samples were applied into the equipment. The LTQ mass spectrometer was operated with m/z from 400-2000 followed by data-dependent MS/MS scans on the six most intense ions with dynamic exclusion enabled. FT target value was 600,000.

H. Data Processing

The acquired MS2 data was extracted and searched using the SEQUEST algorithm (Ver. 28, rev. 13, University of Washington, Seattle, Wash.) in BioWorks (Ver. 3.1, Thermo Fisher Scientific, Waltham, Mass.) against the human and mouse UniRef 100 (Ver. November, 2007) protein database combined with a reverse database and a list of common contaminates for xenograft analysis. Secretome data were searched against without mouse database (human database's version was April, 2010) containing bovine insulin and mouse EGF. Reverse database searching resulted in a specific false positive rate (FDR) of 0.6-3.3% for proteins identified by two or more peptides. The FDR for the dataset was calculated as number of non-redundant reversed sequence hits/number of non-redundant forward sequence hits. Database search and results filtering strategies that we previously optimized for complex proteomes were used (Wang et al, 2010, cited above). Specifically, DTA files were created from raw data using an intensity threshold of 1000 and minimum ion count of 25. The database was indexed with the following parameters: monoisotopic mass range of 800 to 4500, length of 6 to 100, partial tryptic cleavages with a maximum of two internal missed cleavage sites, static modification of cysteine by iodoacetamide (+57.0215 Da) for 2-D secretome and 3-D xenograft analysis or by dimethylacrylamide (+99.0684 Da) for 4-D xenograft method. The data were searched against the indexing database using partial trypsin specificity with a 100 ppm precursor mass tolerance and 1 Da fragment ion mass tolerance. Methionine oxidation and asparagine deamidation were allowed as variable modification. Cysteine carbamidomethylation was also considered as static modification and three different protein translational modifications per peptide were also permitted.

Consensus protein lists were generated by DTASelect (Ver 2.0, licensed from Scripps Research Institute, La Jolla, Calif.) after applying the following filters: full tryptic boundaries, 10 ppm, ΔCn≧0.05. For the purpose of protein and peptide counts, additional Java scripts were developed to compress the non-redundant protein identifications into the smallest sets. Peptide counts were derived after collapsing different forms (charge states and modifications) of the same peptide into a single hit. Further reduction was applied by allowing a peptide to be assigned only once to the protein that has the highest sequence coverage.

In-house software was also developed to separate mouse and human proteins based on their sequence identifiers. Putative human peptides were then searched using BLAST against a comprehensive mouse only database generated by merging all non-identical mouse entries in the Uniref 100. To identify common and unique proteins found by different fractionation methods, protein and peptide data were put into a relational database (MySQL) and matched using custom software. The comparison of common protein and peptide was performed with proteins identified by two and more nonredundant peptides.

To classify the gene/protein function, WebGestalt (WEB-based GEne SeT AnaLysis Toolkit, developed by Vanderbilt University, Nashville, Tenn.) was conducted with common proteins between 2-D_{—}88 secretome and 4-D_{—}175 xenograft analysis.

I. Human Serum.

Human sera from patients with 25 early-stage CRC and 25 late-stage CRC patients were collected before clinical treatment at approximately the time of diagnosis, and control serum samples were collected from healthy, case matched subjects. All patient specimens were collected with full patient consent and in accordance with Institutional Review Board (IRB) and Health Insurance Portability and Accountability Act (HIPAA) requirements.

Human serum samples (typically 30-60 μL) were depleted of the 20-most-abundant serum proteins using a ProteoPrep20 Immunodepletion Column (Sigma), as described previously.19

### Example 2

### Workflow Comparison to Select Effective Method for Cancer Biomarker Discovery

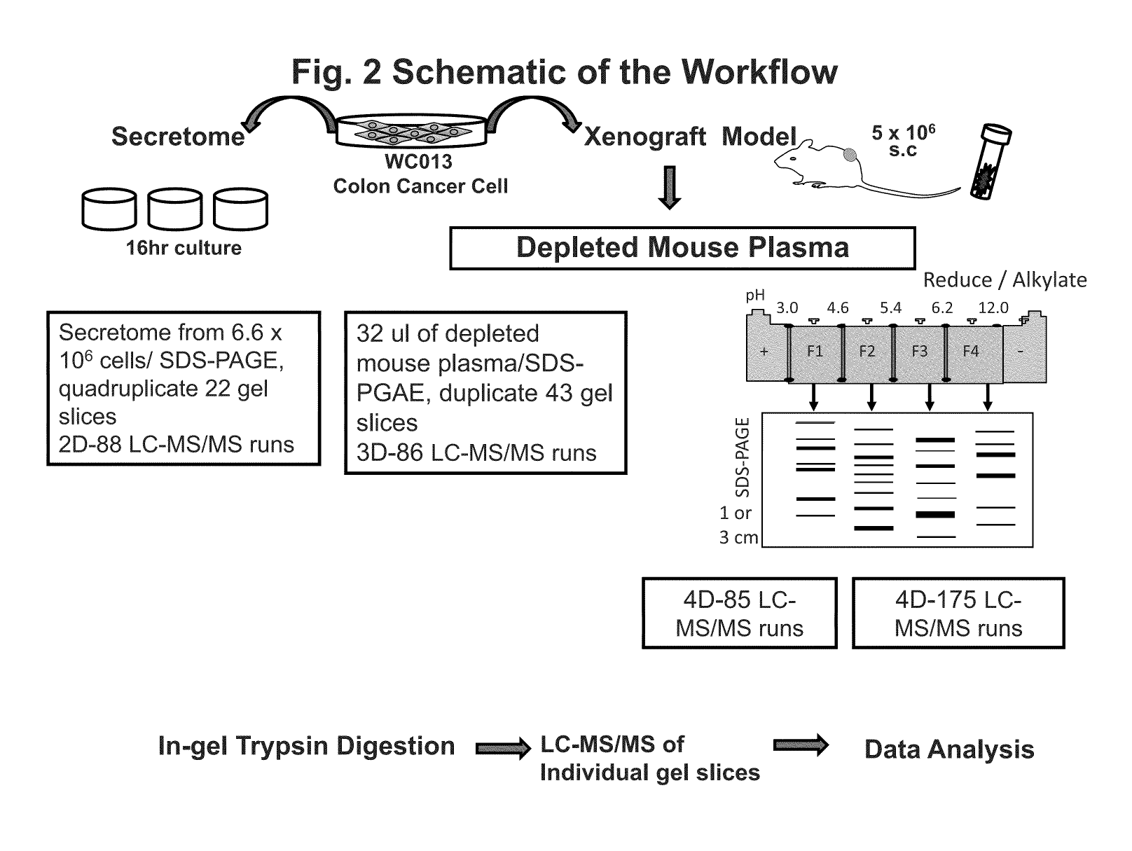

Two model systems, cell secretome and xenograft mouse model, were systematically compared to evaluate the appropriateness of their use as summarized in FIG. 2. To directly evaluate the two model systems, the same early passage human CRC cell line, WC013 was used to compare proteins shedding into the medium in cell culture and shedding into blood in a xenograft mouse model system. For cell secretome (2-D) analysis, the serum-free incubation time was optimized. Minimal cell death (<10%) was observed up to 24 h (data not shown) in cells cultured with serum-free media.

To maximize protein secretion shed by cancer cells as well as to diminish cell death, cell secretome was obtained after 16 h incubation with serum-free media. The conditioned media was analyzed using 1-D SDS gels followed by LC-MS/MS analysis (GeLC-MS/MS) with either duplicate or quadruplicate analysis of 22 gel slices. The xenograft mouse model was performed by either a 3-D method where immmuodepletion of abundant plasma proteins preceded GeLC-MS/MS or a 4-D method where immmuodepletion and microscale solution IEF separation of proteins preceded GeLC-MS/MS. Because one of the goals of the current study was to evaluate the effects of fractionation number, 4-D method was split into two different analyses with either 85 slices (pixels) or 175 pixels. To compare the impact on proteome coverage of each method, mostly equal numbers of LC-MS/MS runs were performed in 2-D_{—}88, 3-D_{—}86, and 4-D_{—}85 method and equal volume of the identical depleted mouse plasma was used in 4-D_{—}85 and 4-D_{—}175 method. All other experimental parameters were maintained as constant as possible.

### Example 3

### Protein Separation of Secretome and 4-D Method

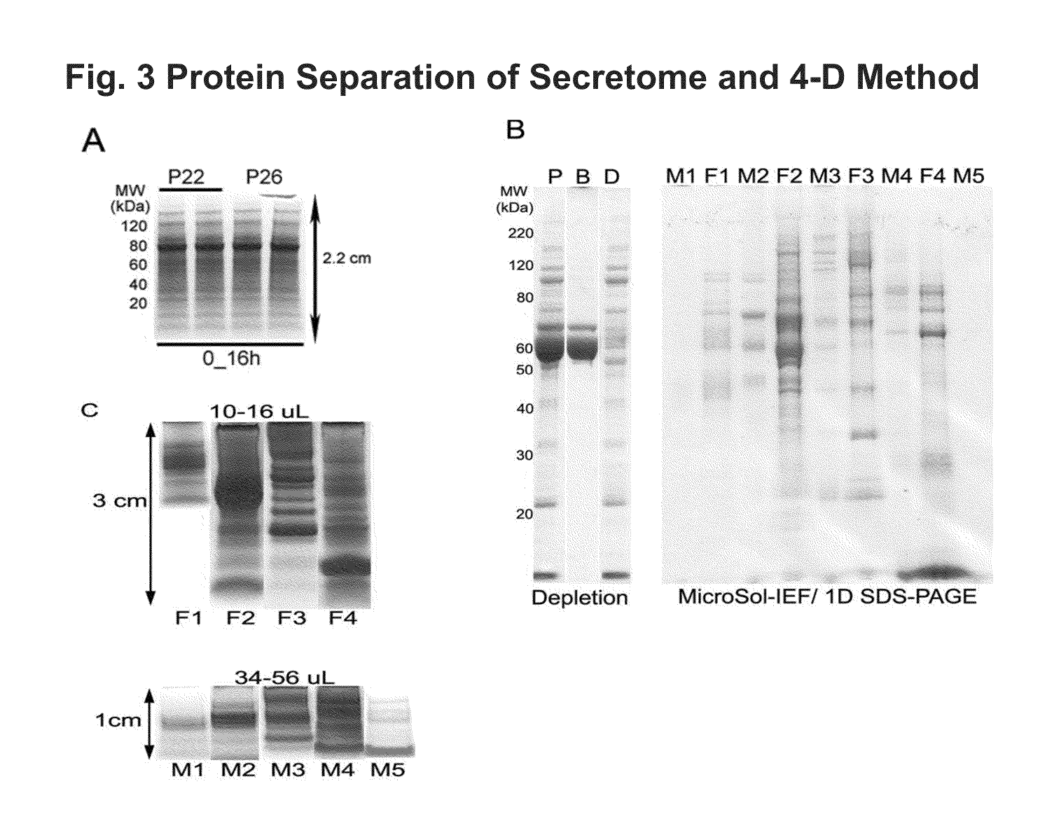

Cell secretome was separated in 1-D gel without any protein pre-fractionation (FIG. 3A). For GeLC-MS/MS analysis of cell secretome, secretome proteins induced from 3.3×10⁶ cells, which was close to a maximum load while avoiding band distortion, were loaded into each of two lanes of a 1D-gel. In order to control total LC-MS/MS analysis time, cell secretome proteins were separated until the tracking dye migrated approximately 22 mm. After staining the gel with Colloidal Coomassie, the 22 mm lane was divided into 22 equal fractions and digested with trypsin. Any difference of protein separation from two biological duplicates of cell secretome was not detected in this study. Hence, 6.6×10⁶ cells of secretome were fractionated into 22 pixels. In 4-D_{—}85 and 4D_{—}175 method, each depleted mouse plasma protein (237 ul) was fractionated for in-depth plasma proteome analysis and detection of low-abundance proteins. Typical results for depletion of major proteins from plasma and fractionation of depleted plasma proteins of 4-D method are shown in FIG. 3B. Extraction from pH membranes located between separation fractions contained a mixture of proteins unique to that membrane and proteins present in adjacent fractions. To maximize protein loading onto the lane of gel, each fraction and membrane extract was concentrated as described in Example 1. FIG. 3C shows concentrated proteins separation for GeLC-MS/MS in 4-D_{—}175 analysis. After MicroSol IEF fractionation, the plasma proteome shed by tumor was divided into four fractions of similar complexity and five membrane extractions in 4-D_{—}175 method. As described in Example 1, the gel lanes from the MicroSol fractions and membrane extractions were cut and digested. Thus, depleted mouse plasma was fractionated into 175 pixels.

### Example 4

### Comparison of Human Protein identification and Peptide Coverage

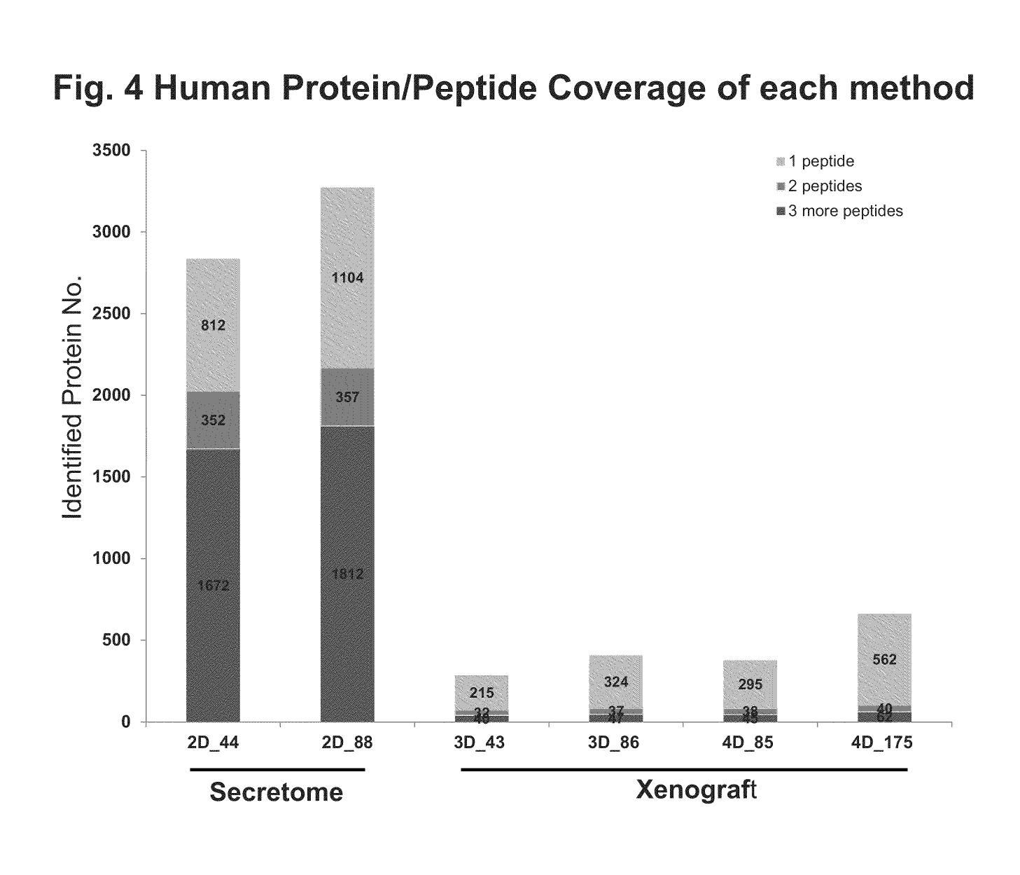

All data were analyzed and filtered as described in Example 1, which resulted in the following protein (identified by two or more peptides) false-positive rates (FDR): 0.6% for cell secretome (2-D), 0.8% for 3-D_{—}86, 0.8% for 4-D_{—}85, and 3.3% for 4-D_{—}175 analysis. Single hit proteins were excluded because we found FDR for these proteins is nearly 40%. FIG. 4 represents the human protein counts and peptide coverage in each method. Forty four analyses of 2-D method were performed by repeat LC-MS/MS runs of duplicates of 22 pixels. The total number of proteins increased with additional replicate data sets for each depth of analysis. The proteins identified by two or more non-redundant peptides increased moderately in intramural comparison of each depth analysis, 7%, 17%, and 23% for 2-D, 3-D, and 4-D, respectively. However, the proteins identified by single hit dramatically increased, 36%, 51%, and 91%, in each depth of analysis. Therefore, fractionation number correlated with discovery of low abundance plasma proteins, because the complex proteome (e.g. plasma) could be effectively separated and low abundance proteins could be easily detected in LC-MS/MS. The comparison of identified protein number and peptide coverage between 3-D_{—}86 and 4-D_{—}85 method showed no significant difference.

To discover colon cancer specific proteins, we investigated the common proteins among four different levels of depth analysis: cell secretome (2-D) vs 3-D_{—}86 vs 4-D_{—}85 vs 4-D_{—}175. All comparisons were performed with proteins identified by two or more nonredundant peptides. The 22 proteins that appeared to be common in all four methods list in FIG. 1. The specific proteins for colon cancer were categorized into medium abundance plasma proteins, high abundance plasma proteins, and candidate biomarkers because reliable and practical cancer biomarkers have been considered as low abundance plasma proteins. The concentration of biomarkers considered viable candidates were lower than 0.1 ug/ml or not detected in healthy control.

### Example 5

### Reproducibility of Mass Analysis in Cell Secretome and Xenograft

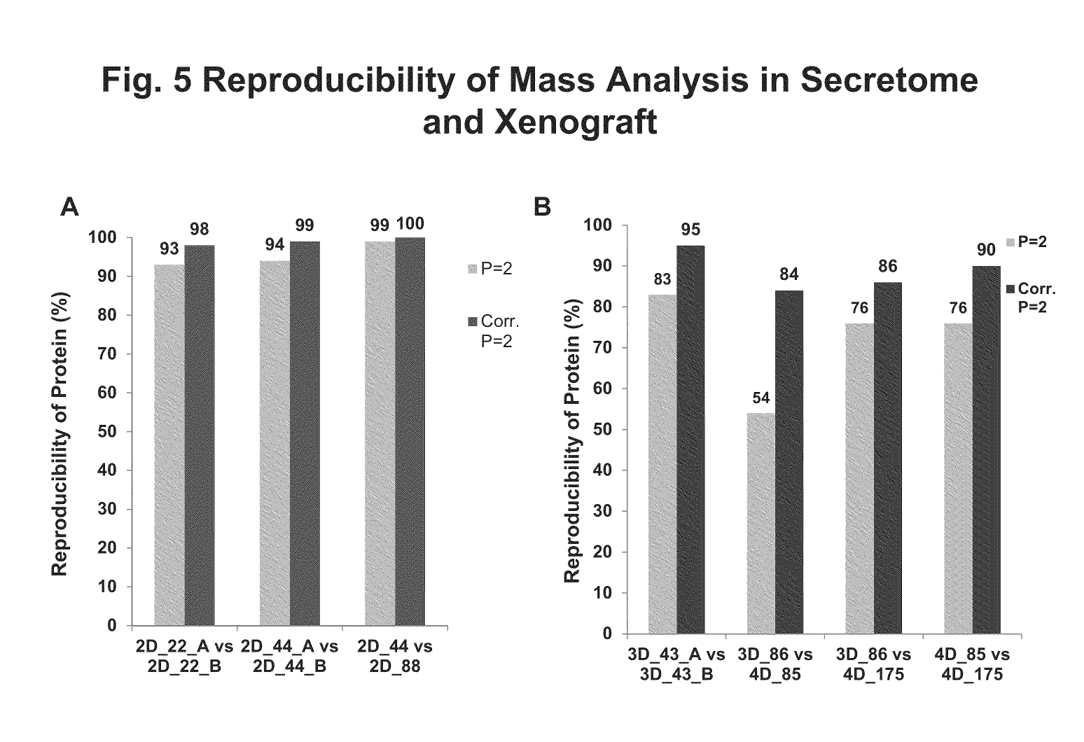

To address how each method is an effective strategy for cancer biomarker discovery, we investigated the reproducibility of mass spectrometer in two model system. These analyses were performed to compare lists of proteins identified by two or more nonredundant peptides in first level comparison (P=2). Further comparison (Corrected P=2) was considered whether unique proteins of smaller data set present in the other larger data set as single-hit proteins. Because the dynamic range of protein concentration of cell secretome was substantially less than in plasma, 2-D method showed an outrageous reproducibility regardless of differing numbers of replicates for mass analysis (FIG. 5A). The repeat runs of 3-D_{—}43 showed the highest reproducibility (95%) and the reproducibility between 3-D_{—}86 and 4-D_{—}85 represented the lowest one as 84% in xenograft model (FIG. 5B).

### Example 6

### Common Proteins in Cell Secretome and Xenograft

The proteome produced by the cell secretome/four repetitive-run (88 pixels) and the 4-D_{—}175 methods were compared using the same strategy for assessing the reproducibility of mass spectrometer, as described above. This comparison was facilitated since both data sets were searched using the same database (UnirRef 100) and DTASelect lists all protein names identified in the search. Two data sets shared 60 proteins, which corresponded to 60% of the smaller 4-D_{—}175 proteome in first level comparison. No matter of further comparison (corrected P=2), whether unique 42 proteins of 4-D_{—}175 present in the cell secretome_{—}88 set as single-hit proteins, overlap percentage did not change (data not shown).

To classify of gene function, common 60 proteins were categorized in ten different molecular functions in. Protein binding, ion binding, and transferase activity were the 3 major functions in these common proteins between cell secretome and 4-D_{—}175 method.

### Example 7

### Quantitation of Peptides

The detected peptides from the 22 proteins discussed in Example 5, are listed in FIG. 1. To ensure that quantitation of peptides in human serum was optimal, we evaluated linearity of response and limit of quantification (LOQ) using “heavy” stable isotope labeled synthetic peptide standards. All synthetic peptides were injected onto the LC-triple quadrupole mass spectrometer system over a range of concentrations spanning 0.5 to 13800 attomoles. The amounts of standards spiked into patient plasma fractions were 3, 6, or 10 fmol based on peptide intensity. The results showed that the majority of peptides utilized in the MRM assay exhibited good linearity over an acceptable concentration range (R²>0.96). The exceptions were two peptides (LEEQRPER and DGLEM[oxi]EK for TPT1, which were therefore excluded from quantitative analysis. In addition, one peptide (FVFDRPLPVSR of PSMA1) exhibited a linear range of 0.5-800 atmol, whereas the highest value observed in plasma samples was 832 atmol. However, this difference was minor and was not expected to significantly affect quantitation of the protein.

### Example 8

### Analysis of Human Serum

MRM analyses were performed as described above on all of the patient and control serum samples. Values were calculated for all samples using only reliably quantitated peptides. Femtomole values were converted to estimated pg/ml concentrations based upon amounts of peptides spiked into samples, MW, and volumes analyzed. It should be noted that these concentrations are approximate because the internal standard peptides had varying purity and their precise concentrations were not accurately determined by either the vendor or us. However, relative amounts should be internally consistent.

Results are reported in FIGS. 6-9 in scatterplots showing three comparisons for each candidate biomarker. First, values for the 25 normal subjects were compared to the 25 advanced cancer patients. In addition, the 25 normal samples were compared to the subsets of advanced cancer samples that had either CEA or CA19-9 values below the average of CEA or CA19-9, respectively, in the entire late cancer group. This was used as an initial test to evaluate whether some of the biomarkers might complement CEA and CA19-9 in a multi-biomarker panel.

The candidate biomarkers were divided into four groups (FIGS. 6-9). FIG. 6 shows data for the four biomarkers (LMNB1, ECI1, NME2 and CALR) that exhibit the largest differences between normal and late stage CRC patients. These biomarkers compare very favorably with CEA and CA19-9 values from ELISA assays using the same set of 50 patients. Furthermore, preliminary evaluation strongly suggests that these four proteins could complement CEA and CA19-9 because most low CEA and low CA19-9 patients have levels of these biomarkers that are higher than controls. Also, these biomarkers are substantially superior to the EDRN biomarker, TIMP1.

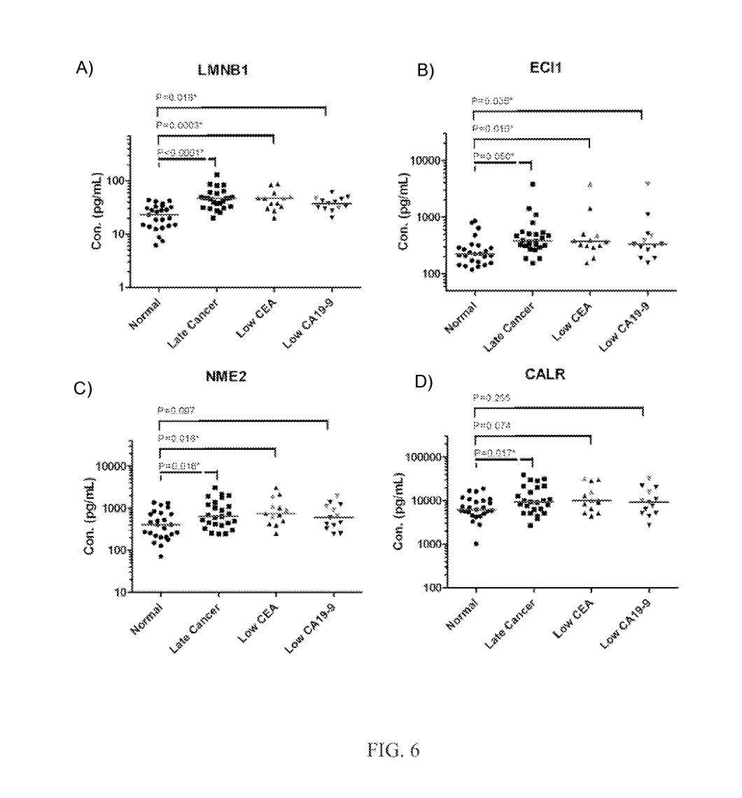

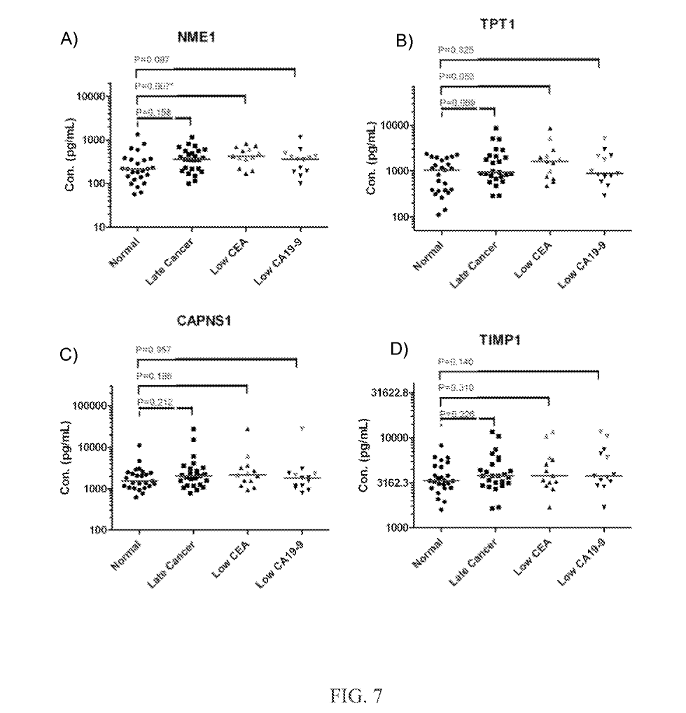

FIG. 7 shows data for an additional four biomarkers, including the EDRN biomarker, TIMP1. These candidates are elevated on average in late cancer but do not show significant differences between normal subjects and late cancer. However, they might show better performance in larger patient cohorts or in combination with other biomarkers. In particular, it is interesting that NME1 and TPT1 exhibit significant and nearly significant differences, respectively, between normal and low CEA. Furthermore, TPT1 is close to significant (p=0.069) for the 25 normal vs. 25 late cancer comparison.

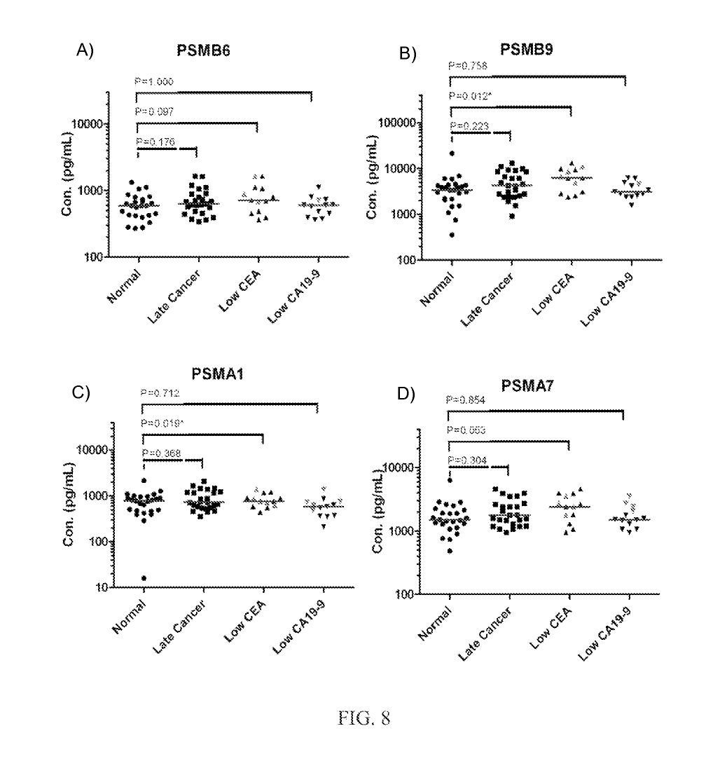

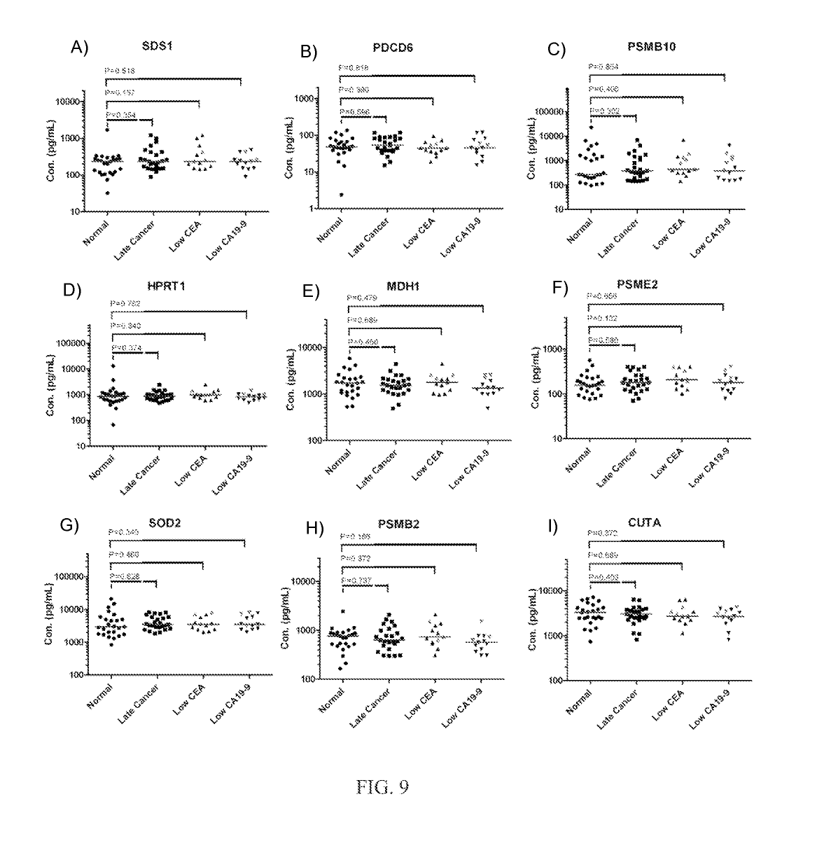

FIG. 8 shows data for four additional biomarkers that are proteosome subunits. The distinction between this group and those in FIG. 7 is largely subjective and based on differences in biology. That is, they are not significantly elevated in the full advanced cancer cohort but are significant or close to significant compared with the low CEA group. Interestingly, while these subunits show elevated levels in the low CEA samples, other proteosome subunits show not differences between groups of patients (see FIG. 9).

Finally, the remaining nine biomarker candidates do not show any promising differences between cancer and control samples. Hence they are probably not worth pursuing further (FIG. 9).

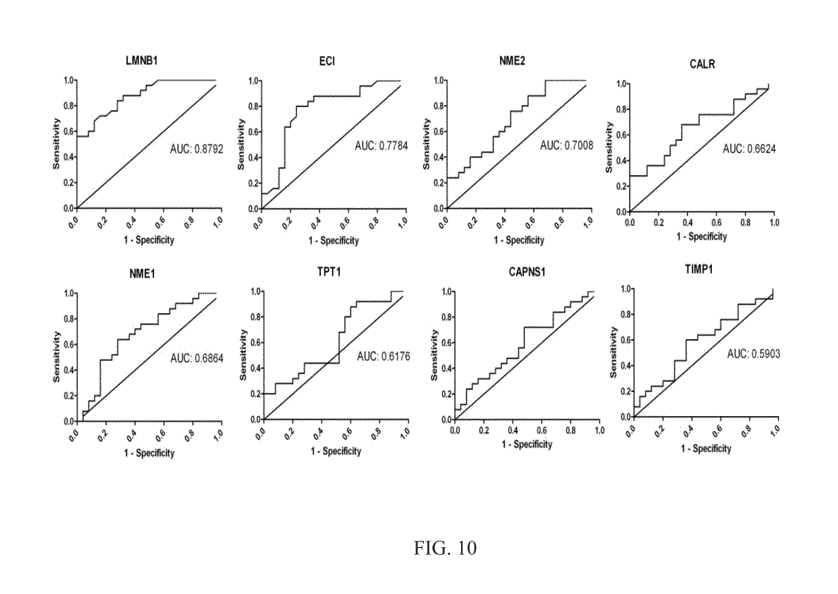

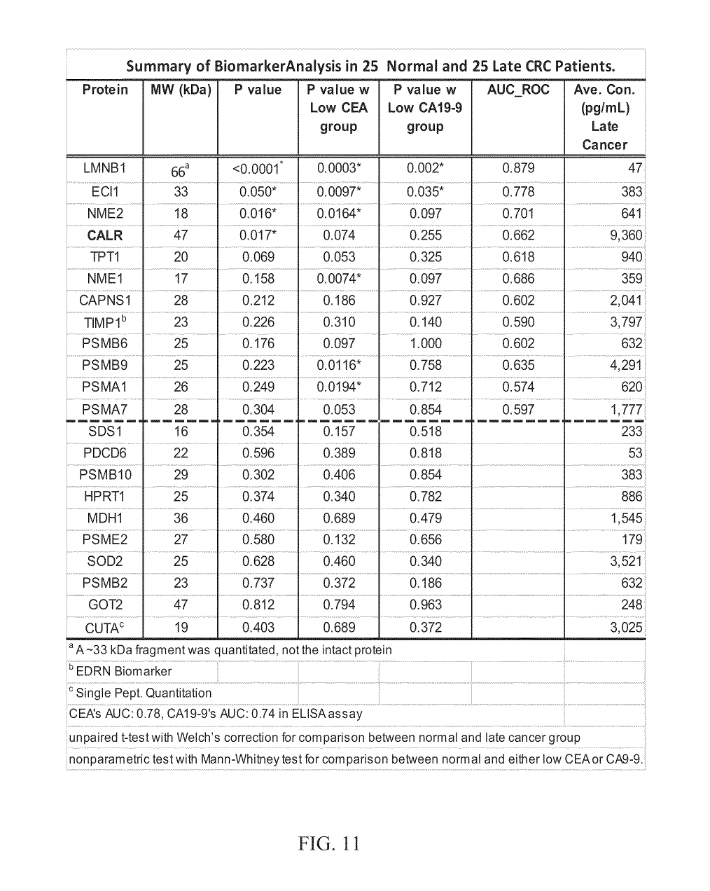

Receiver operator curves for the most promising biomarkers are shown in FIG. 10. Data for all biomarker candidates are summarized in FIG. 11. Interestingly, as noted above, some biomarkers such as NME1, PSMB9 and PSMA1 showed significant differences (p<0.05) between the normal group and the low CEA subgroup of the late cancer patients, although there was no difference between the normal group and all late cancer patients.

The 11 biomarkers above the line in FIG. 11 are valuable CRC biomarkers (not including the ERDN biomarker, TIMP1). Based upon available data, LMNB1, ECI1, and NME2 are the most significant. In this patient cohort, these three biomarkers compare favorably in terms of sensitivity and specificity to CEA and CA19-9. In addition, CALR and NME1 are very promising and NME1 is related to NME2, suggesting that combined analysis of these two proteins may provide an advantage.

Each of the documents cited herein, as well as priority U.S. Provisional Application No. 61/775,501, is incorporated herein by reference in its entirety.

What is claimed is:

1. A diagnostic reagent comprising at least one ligand capable of specifically complexing with, binding to, or quantitatively detecting or identifying a single target biomarker selected from the group consisting of:
a. lamin B1 (LMNB1);
b. enoyl-CoA delta isomerase 1 (ECI1, DCI);
c. NME/NM23 nucleoside diphosphate kinase 2 (NME2);
d. Calreticulin (CALR); and
e. an isoform, pro-form, modified molecular form, or peptide fragment of any of biomarkers (a) through (d), proteins in the same biomarker family or expressed from a related gene, having at least 90% sequence homology or sequence identity with any biomarker (a) through (d);
wherein at least one ligand is associated with a detectable label or with a substrate.

2. The diagnostic according to claim 1, further comprising at least one additional ligand capable of specifically complexing with, binding to, or quantitatively detecting or identifying a single target biomarker selected from the group consisting of:
f. proteasome (prosome, macropain) subunit, beta type 6 (PSMB6);
g. proteasome (prosome, macropain) subunit, beta type 9 (large multifunctional peptidase 2) (PSMB9);
h. proteasome (prosome, macropain) subunit, alpha type, 1 (PSMA1);
i. tumor protein, translationally-controlled 1 (TPT1);
j. NME/NM23 nucleoside diphosphate kinase 1 (NME1);
k. proteasome (prosome, macropain) subunit, alpha type, 7 (PSMA7); and
l. an isoform, pro-form, modified molecular form, or peptide fragment of any of biomarkers (f) through (k), proteins in the same biomarker family or expressed from a related gene, having at least 90% sequence homology or sequence identity with any biomarker (f) through (k);
wherein at least one ligand is associated with a detectable label or with a substrate.

3. The reagent according to claim 2, comprising multiple ligands selected from (a) through (l), each ligand directed to a different biomarker.

4. The reagent according to claim 1, comprising each of ligands (a)-(d).

5. The reagent according to claim 1, further comprising at least one ligand that specifically complexes with, binds to, quantitatively detects or identifies the biomarker, Carcinoembryonic Antigen (CEA) or Carbohydrate Antigen 19-9 (CA19-9), or an isoform, pro-form, modified molecular form, or peptide fragment therefrom.

6. The reagent according to claim 1, wherein each said ligand is selected from an antibody or fragment of an antibody, antibody mimic or equivalent that binds to or complexes with a target biomarker.

7. The reagent according to claim 1, wherein one or more ligands are immobilized on a substrate, each ligand specifically complexing with, binding to, quantitatively detecting or identifying a different biomarker selected from (a) to (e).

8. A kit, panel or microarray comprising at least two diagnostic reagents of claim 1, each reagent identifying a different biomarker.

9. A method for diagnosing or detecting or monitoring the progress of colorectal cancer in a subject comprising:
(i) contacting a sample obtained from a test subject with the diagnostic reagent of claim 1;
(ii) detecting or measuring in the sample or from a protein level profile generated from the sample, the protein levels of one or more of the biomarkers (a) to (e), or ratios thereof;
(iii) comparing the protein levels of the biomarker in the subject's sample or from a protein level profile or ratio of multiple said biomarkers, with the level of the same biomarker or biomarkers in a reference standard;
(iv) diagnosing, detecting or monitoring the progress of colorectal cancer in the subject based on a significant change in the protein level of the subject's sample biomarker or biomarkers from that in the reference standard.

10. The method according to claim 9, wherein the reference standard is selected from:
(I) a reference human subject or a population of subjects having no colorectal cancer;
(II) a reference human subject or a population of subjects having benign colorectal polyps;
(III) a reference human subject or a population of subjects following surgical removal of a colorectal tumor;
(IV) a reference human subject or a population of subjects prior to surgical removal of a colorectal tumor;
(V) a reference human subject or a population of subjects following therapeutic treatment for a colorectal tumor;
(VI) a reference human subject or a population of subjects prior to therapeutic treatment for a colorectal tumor;
(VII) the same subject who provided a temporally earlier biological sample;
(VIII) a reference human subject or a population of subjects without colorectal cancer but which tests positive for a protein level of CEA;
(IX) a reference human subject or a population of subjects with colorectal cancer but which tests negative for a protein level of CEA;
(X) a reference human subject or a population of subjects without colorectal cancer but which tests positive for a protein level of CA19-9;
(XI) a reference human subject or a population of subjects with colorectal cancer but which tests negative for a protein level of CA19-9;
(XII) a reference human subject or a population of subjects having early stage colorectal cancer; and
(XIII) a reference human subject or a population of subjects having advanced stage colorectal cancer.

11. The method according to claim 9, wherein said change in protein level of each said biomarker comprises an increase in comparison to said reference or control.

12. The method according to claim 9, wherein said diagnosis or detecting comprises early diagnosis of disease, determining the best clinical treatment, monitoring relapse after initial diagnosis and treatment, or predicting clinical outcome.

13. The method according claim 9, wherein the biological sample is selected from group consisting of whole blood, plasma, serum, fecal matter, circulating tumor cells, tumor secretome fluid, urine and tumor tissue.

14. The method according to claim 13, wherein the biological sample is plasma.

15. The method according to claim 9, comprising performing a serum or plasma ELISA, sandwich ELISA, or equivalent assay, RT-PCR, any assay capable of quantifying the target protein or peptides thereof, or performing a mass spectrometry-based test.

16. The method according claim 9, wherein the biomarker or biomarkers are present in different levels or abundance profiles in biological samples of two or more of the conditions selected from:
(1) no colorectal cancer;
(2) benign colorectal polyps;
(4) following surgical removal of a colorectal tumor or cells;
(5) prior to surgical removal of a colorectal tumor or cells;
(6) following therapeutic treatment for a colorectal cancer;
(7) periodically during treatment for colorectal cancer;
(8) prior to therapeutic treatment for colorectal cancer; and
(9) undiagnosed clinical symptoms of unknown origin selected from abdominal pain or abdominal tenderness; blood in the stool; diarrhea, constipation, or other change in bowel habits; narrow stools; or weight loss;
(10) early stage colorectal cancer; and
(11) advanced stage colorectal cancer.

17. A method of diagnosing, or detecting a risk of developing, a colorectal cancer in a subject comprising:
(a) contacting a sample obtained from a test subject with a composition of claim 1;
(b) detecting or measuring in the sample or from an expression profile generated from the sample, the expression levels of one or more of the target biomarkers, or ratios thereof;
(c) comparing the expression levels of the biomarker in the subject's sample or from an expression level profile or ratio of multiple said biomarkers, with the level of the same biomarker or biomarkers in a reference standard;
wherein a significant change in expression level of the subject's sample biomarker or biomarkers from that in the reference standard indicates a diagnosis, risk, or the status of progression or remission of colorectal cancer in the subject.

18. The method according to claim 17, wherein the sample is a biopsy sample, surgical sample, or tumor cell sample.

19. A diagnostic reagent comprising at least one ligand capable of specifically complexing with, binding to, or quantitatively detecting or identifying a single target biomarker selected from the group consisting of:
a. lamin B1 (LMNB1);
b. enoyl-CoA delta isomerase 1 (ECI1, DCI);
c. NME/NM23 nucleoside diphosphate kinase 2 (NME2);
d. Calreticulin (CALR);
e. tumor protein, translationally-controlled 1 (TPT1);
f. NME/NM23 nucleoside diphosphate kinase 1 (NME1); and
g. an isoform, pro-form, modified molecular form, or peptide fragment of any of biomarkers (a) through (f), proteins in the same biomarker family or expressed from a related gene, having at least 20% sequence homology or sequence identity with any biomarker (a) through (f);
wherein at least one ligand is associated with a detectable label or with a substrate.

20. The diagnostic according to claim 19, further comprising at least one additional ligand capable of specifically complexing with, binding to, or quantitatively detecting or identifying a single target biomarker selected from the group consisting of:
h. proteasome (prosome, macropain) subunit, alpha type, 7 (PSMA7);
i. proteasome (prosome, macropain) subunit, beta type 6 (PSMB6);
j. proteasome (prosome, macropain) subunit, beta type 9 (large multifunctional peptidase 2) (PSMB9);
k. proteasome (prosome, macropain) subunit, alpha type, 1 (PSMA1); and
l. an isoform, pro-form, modified molecular form, or peptide fragment of any of biomarkers (h) through (k), proteins in the same biomarker family or expressed from a related gene, having at least 20% sequence homology or sequence identity with any biomarker (h) through (k);
wherein at least one ligand is associated with a detectable label or with a substrate.
